(19) United States
(12) Patent Application Publication
(10) Pub. No.: US 2005/0038454 A1
(43) Pub. Date: Feb. 17, 2005

(54) Anastomotic delivery system

(75) Inventors: Amir Loshakove, Moshav-Bazra (IL); Ido Kilemnik, Herzelia (IL); Gil Hefer, Kfar-Saba (IL); Aharon Shlomo Friedman, Haifa (IL); Ofer Nativ, Rishon-Lezion (IL)

Correspondence Address: William H. Dippert, Esq.;c/o Reed Smith LLP, 29th Floor, 599 Lexington Avenue, New York, NY, 10022-7650, US

(73) Assignee: By-Pass, Inc., Orangeburg, NY (US)

(21) Appl. No.: 10/809,274

(22) Filed: Mar. 25, 2004

Related U.S. Application Data
(63) Continuation of application No. PCT/IL02/00790, filed on Sep. 25, 2002.
(63) Continuation-in-part of application No. 10/668,059, filed on Sep. 22, 2003.
Application No. 10/668,059 is a continuation of application No. PCT/IL02/00215, filed on Mar. 18, 2002.
(63) Continuation-in-part of application No. 10/459,373, filed on Jun. 11, 2003.
Application No. 10/459,373 is a continuation of application No. PCT/IL01/01019, filed on Nov. 4, 2001.
(63) Continuation-in-part of application No. 10/402,375, filed on Mar. 27, 2003.
Application No. 10/402,375 is a continuation of application No. PCT/IL01/00903, filed on Sep. 25, 2001.
(63) Continuation-in-part of application No. 10/459,407, filed on Jun. 11, 2003.
Application No. 10/459,407 is a continuation of application No. PCT/IL01/00600, filed on Jun. 28, 2001.
(63) Continuation-in-part of application No. 10/239,387, filed on May 30, 2003.
(63) Continuation-in-part of application No. 10/239,364, filed on May 15, 2003.
(63) Continuation-in-part of application No. 10/239,365, filed on Jun. 2, 2003.
(63) Continuation-in-part of application No. 10/221,982, filed on Sep. 18, 2002.
(63) Continuation-in-part of application No. 10/149,302, filed on Oct. 23, 2002.
(63) Continuation-in-part of application No. 10/149,364, filed on Oct. 7, 2002.
(63) Continuation-in-part of application No. 09/936,805, filed on Sep. 17, 2001.
(63) Continuation-in-part of application No. 09/936,796, filed on Sep. 17, 2001, now Pat. No. 6,726,704.
(63) Continuation-in-part of application No. 09/936,789, filed on Sep. 17, 2001.
(63) Continuation-in-part of application No. 09/936,806, filed on Sep. 17, 2001.
(63) Continuation-in-part of application No. 09/701,523, filed on Nov. 28, 2000.
(63) Continuation-in-part of application No. 09/701,531, filed on Nov. 28, 2000.

(30) Foreign Application Priority Data
May 29, 1998 (IL) 124694
Mar. 19, 1999 (IL) 129067
Jun. 28, 2001 (IL) 144051
Sep. 25, 2001 (WO) PCT/IL01/00903

(51) Int. Cl. C12Q 1/68; A61B 17/08

(52) U.S. Cl. 606/153; 606/151

(57) ABSTRACT

An anastomotic connector (200) for attaching two blood vessels comprising a plurality of eye segments (1604), each defining a channel and each including a part of an interlock mechanism (1600) on said channel; a plurality of hook segments (1602), each defining a tissue holding area, each adapted to pass through said channel and including a second part of said interlock mechanism (1600), wherein, said interlock mechanism (1600) engages for a hook and an eye segment when said hook segment (1602) is retracted back into said eye segment (1604) enough to attach two layers of vascular tissue between said eye segment (1604) and said hook segment (1602).

### RELATED APPLICATIONS

The present application is a continuation in part of PCT application PCT/IL01/00903, filed on Sep. 25, 2001, which designates the U.S., now published in English as WO 02/30172, the disclosure of which is incorporated herein by reference. This application is also a continuation-in-part of PCT/IL02/00215 filed on Mar. 18, 2002, PCT/IL01/01019, filed on Nov. 4, 2001, PCT/IL01/00903, filed on Sep. 25, 2001, PCT/IL01/00600, filed on Jun. 28, 2001, PCT/IL01/00267, filed on Mar. 20, 2001, PCT/IL01/00266, filed on Mar. 20, 2001, PCT/IL01/00074, filed on Jan. 25, 2001, PCT/IL01/00069, filed on Jan. 24, 2001, PCT/IL00/00611, filed on Sep. 28, 2000, PCT/IL00/00609, filed on Sep. 28, 2000, PCT/IB00/00310, filed on Mar. 20, 2000, PCT/IB00/00302, filed on Mar. 20, 2000, PCT/IL99/00674, filed on Dec. 9, 1999, PCT/IL99/00670 filed on Dec. 8, 1999, PCT/IL99/00285, filed on May 30, 1999, and PCT/IL99/00284, filed on May 30, 1999. The disclosure of all of these applications, which designate the U.S. and were filed in English, are incorporated herein by reference.

### FIELD OF THE INVENTION

The present invention relates to anastomotic connector and delivery systems therefor.

### BACKGROUND OF THE INVENTION

Anastomotic connectors that connect a grafted vessel with a host vessel, for example an interior mammary artery with a coronary artery, are known in the art. An exemplary embodiment of a coronary anastomotic connector and a method of deployment are shown in FIGS. 1A-1G, corresponding to FIGS. 12A-12G of PCT publication WO 01/70090, the disclosure of which is incorporated herein by reference. The FIGS. have been amended to show the blood vessels.

A brief review of FIGS. 1A-1G is presented herein to provide a background for the present invention, a system for delivering an anastomotic connector and a connector for such a system.

FIG. 1A is an anastomotic connector base 100, comprising a ring 102 having a plurality of apertures 103 through which a plurality of spikes 228 (FIG. 1B) pass. As shown, each aperture 103 comprises a leaf spring section 106 for engaging and/or stabilizing spikes 228.

FIGS. 1B-1D demonstrate deployment of an anastomotic connector 101 with a hub 104 to which spikes 228 are attached. Each spike 228 extends through ring 102 and ends in a hook 220. A graft vessel 140 is shown everted on hooks 220. Then, hooks 220 are aligned with an aperture 143 in a target.

In FIG. 1C, hub 104 is retracted in a direction 142 in relation to ring 102 so that hooks 220 are pulled toward ring 102 and engage the lips of aperture 143 in vessel 144. As hooks 220 are pulled in direction 142 (FIG. 1B), spikes 228 expand radially outward, attaining an expanded configuration as seen in FIG. 1C. Possibly, radial expansion of hooks 220 aids in extending aperture 143.

In FIG. 1D, spikes 228 are severed from hooks 220. Spikes 228 and hub 104 are then removed from blood vessels 140 and 144.

FIGS. 1E and 1F demonstrate the process by which a single spike 228 is severed from a single hook 220 during the above-noted anastomotic connection. FIG. 1E shows hook 220 attached to spike 228 at a weakened area 120. Also shown is a extension 114, not shown in FIGS. 1A-1D.

In FIG. 1F, spike 228 is pulled in direction 142 and extension 114 is stabilized against a stopper 118 so that hook 220 remains stationary. Spike 228 severs from hook 220 at weakened area 120. Typically, hooks 220 and ring 102 remain in place within the anastomosed tissue while grafted vessel 140 and host vessel 144 heal together properly (FIG. 1G).

An anastomotic connection between grafted vessel 140 and host blood vessel 144, for example between an interior mammary artery and a coronary artery, is a delicate procedure. There may be situations, for example in making an anastomotic connection in an adolescent or child, where the chest cavity is narrow. In narrow areas, hooks 220 may be manipulated to an extent that they are pressed, inappropriately, against a biologic tissue with force sufficient to bend, twist or knot hooks 220. While inadvertent damage of hooks 220 is not a common occurrence, it may result in an improper anastomosis between grafted vessel 140 and host vessel 144, a significant problem in a coronary artery upon which the heart depends for a continuous supply of nutrients, even if no leaking occurs. Following are exemplary acts where some difficulty in execution may cause the connection to fail:
(i) cutting the grafted vessel at the correct angle to and make sure it connects at the proper angle with the host vessel;
(ii) properly attaching connector hooks 220 to grafted vessel 140 in preparation for making the anastomotic connection;
(iii) ensuring proper orientation of connector hooks 220 in relation to grafted vessel 140 so the hooks align within the edges of aperture 160 in host vessel 144;
(iv) guiding connector hooks 220 and grafted vessel 140 into aperture 160 in host vessel 144 without inadvertently damaging to the delicate connector 101, its hooks 220 and/or their position in relation to grafted 140 and/or host vessel 144;
(v) freeing hooks 220 from all deployment and/or guidance instrumentation so that hooks properly conform to aperture 160 in host vessel 144; and
(vi) properly attaching and securing each hook 220 of anastomotic connector 101 to host vessel 144 to establish a proper connection to grafted vessel 140.

Typically, a small “keyhole” incision in the chest is made through which the anastomosis procedure is performed. Working through the keyhole incision with the slippery, moving and wet structures of the heart and its milieu, can result in damage to connector 101, hooks 220 and/or an improper anastomosis.

### SUMMARY OF THE INVENTION

An aspect of some embodiments of the invention relates to a blood vessel cutter adapted to form an aperture in a blood vessel suitable for entry and alignment of an anastomotic connector. In an exemplary embodiment, the blood vessel cutter comprises a handle adapted to rotate around a longitudinal axis with a horn shaped cutting edge perpendicularly connected to one end of said handle. In an exemplary embodiment, upon rotating said handle around its longitudinal axis, said cutting edge rotates around said handle. When held proximate to a vessel, for example, by placing the cutting edge along the longitudinal axis of a blood vessel, the cutting edge cuts an aperture into said vessel during rotation of the handle. In an exemplary embodiment of the invention, the horn shape comprises an outside curve and an inside curve, which optionally have the same radius. The tip and at least part of the inner curve are sharp. Alternatively or additionally, the outside curve is sharp. In an exemplary embodiment of the invention, the width of the blade matches a desired incision width. Alternatively or additionally, different widths may be provided by rotating the cutter more. Optionally, a sharp even width section is provided at the tip, for assistance in piercing the blood vessel.

In an exemplary embodiment, said horn-shaped cutting edge is of a length appropriate to cut an aperture in a surface of the blood vessel that allows the hooks to enter the vessel easily and hook into the walls of the vessel around said aperture. Optionally, said aperture is cut in said vessel by said vessel cutter during a single rotation thereof and comprises a longitudinal cut, the length of said cutting edge. Alternatively or additionally, said cutter is rotated multiple times in relation to said vessel and optionally moved longitudinally along said vessel to create said aperture.

A broad aspect of the invention relates to a delivery system for delivering an anastomotic connector to a target vessel.

An aspect of some embodiments of the invention relates to an anastomotic delivery system for delivering an anastomotic connector into a blood vessel and tearing extensions of spikes of the anastomotic connector. In an exemplary embodiment of the invention, the extensions are coupled to a body, to which also a tensioned spring may be coupled. A means for retracting the body, and thus the extensions, is provided. However, when a certain degree of retracting is reached, an interlock locking the spring is released and the spring forcefully retracts the body with a strong force. A potential advantage of this system is that an operator does not apply this force himself and may be less likely to cause inadvertent motion of the delivery system during application of this force. In an exemplary embodiment of the invention, a rotation knob, for example rotated using a thumb, is used to retract the body, for example a tube.

An aspect of some embodiments of the invention relates to using a separable capsule in conjunction with a delivery system. In an exemplary embodiment of the invention, the capsule includes the mounting for an anastomosis connector and for a graft. In an exemplary embodiment of the invention, the capsule includes a mechanism for selectively advancing an retracting spikes of the connector, for example retracting them so that they are shorter and stiffer during eversion. Optionally, the mechanism is a retracting pin. Alternatively or additionally, the mechanism is a relative rotation of two parts of the capsule. In an exemplary embodiment of the invention, when the capsule is inserted into the rest of the delivery system, the rotating mechanism is hidden, so an operator will not inadvertently operate it.

An aspect of some embodiments of the invention relates to a delivery system in which a tip of the delivery system, for example a capsule portion thereof can rotate relative to a handle portion thereof. In an exemplary embodiment, the rotation takes place while the hooks and/or spikes of a connector are being placed and/or being deployed in an aperture in a blood vessel. In an exemplary embodiment, the anastomotic connector is rotated so that its movement and/or the movement of its levers and/or other moving parts, are not restricted in their motion by biologic tissue in the surgical area, for example, portions of a keyhole in a chest.

In an exemplary embodiment, the anastomotic rotating device allows rotation of the body while the spikes and hooks remain stable in position (e.g., held by hand) in an aperture in a blood vessel, for example a coronary artery. Additionally or alternatively, the anastomotic rotating device allows rotation of the grafted vessel, for example the Left Interior Mammary Artery (LIMA) in relation to the aperture in the host vessel. In this fashion, for example, when the LIMA or TMA are cut obliquely to their longitudinal axis, and the rotatable anastomotic connector is held at an oblique angle to the aperture, the cut end of the grafted vessel can be aligned with the aperture.

An aspect of some embodiments of the invention relates to a transfixing assistance device for transfixing a blood vessel on one or more hooks of an anastomotic connector. In an exemplary embodiment, the transfixing device comprises a handle defining a longitudinal axis and an extension offset at an angle to said longitudinal axis. In an exemplary embodiment, when said handle is rotated during said transfixing, the tip of the extension-offset circumducts, for example, according to the curvature of a hook on an anastomotic connector. In an exemplary embodiment, the tip of the extension comprises an orifice (e.g., an aperture or a slot) adapted fit over the hook and to encourage movement of blood vessel tissue in relation to said one or more hooks without damaging the vessel and/or hook during transfixing.

In an alternative embodiment, a transfixing assistance device comprises a transfixing forceps with two legs, one or both legs ending in a transfixing extension with an orifice adapted for encouraging movement of a blood vessel tissue on said one ore more hooks. In an exemplary embodiment of the invention, this forceps is used to evert part of the vessel onto the hook, so that it catches on it and then the orifice is used to complete the transfixing by circumducting.

An aspect of some embodiments of the invention relates to an arrangement device that arranges a plurality of anastomotic hooks during placement in an aperture in a blood vessel. In an exemplary embodiment, the hooks are maintained in a. flat plane or a curved plane or another shape, for example that matches the configuration of the aperture in the blood vessel. Optionally, the hooks are arranged so that no sharp points project in a manner that can inadvertently engage tissue.

In an exemplary embodiment, the anastomotic hook arranging device comprises one or more plates that arrange a plurality of hooks projecting from an anastomotic connector, for example by pressing against said hooks. Optionally, the one or more plates comprise two or more plates. Alternatively or additionally, the one or more plates comprise one or more hook spacers, adapted to space the hooks in relation to each other on said anastomotic connector.

An aspect of some embodiments of the invention relates to an anastomotic hook guide comprising one or more walls for guiding a plurality of anastomotic hooks into a blood vessel. In an exemplary embodiment, the guide is placed within and/or near an aperture in a blood vessel and guides the anastomotic hooks into the vessel, thereby preventing inadvertent damage to the plurality of hooks from contact with the vessel and/or other biologic tissue in the area.

In an exemplary embodiment of the invention, the guide defines a tip that is inserted in the blood vessel and a narrow slot, along which the hooks are guided and which ensures that the hooks are together and do not contact nearby tissue. In an exemplary embodiment of the invention, the guide comprises a slofted tube with a flared entry at one side and a narrower tip at the other. Optionally, the tip is used for piercing and/or forming an incision in the blood vessel.

Optionally, the guide and/or the assistance device are permanently or removably mounted on a delivery system. In an exemplary embodiment of the invention, the guide is rotatably mounted in a plane of the axis of the delivery system.

An aspect of some embodiments of the invention relates to a releasable grasper for grasping a plurality of hooks on an anastomotic connector in a receptacle thereof. In an exemplary embodiment, the grasper comprises a grasping wire loop, which when contracted, compresses a plurality of connector hooks towards each other. Optionally, when the wire is further contracted, for example, by retracting against a tube, the wire is torn and the hooks released. This additional retraction may be practiced, for example, after the hooks are inserted in a blood vessel. The grasper is optionally mounted on a deliver system. Alternatively, it may be a separate device. Alternatively to a tearing wire, one or more bent wires may be used, which when retracted, pull through the tube, leaving the hooks free.

An aspect of some embodiments of the invention relates to a clip for anastomotic connection including an interlock mechanism. In an exemplary embodiment of the invention, the clip comprises an eye segment and a hook segment, where an interlock mechanism is defined, one part on each segment, to interlocks the two segments, using a single interlock mechanism. In an exemplary embodiment of the invention, the interlocking substantially prevents movement in any direction, possibly providing some elasticity or some freedom of motion due to spaces, however.

In an exemplary embodiment of the invention, the interlocking mechanism comprises one or more tabs and one or more matching apertures for the tabs to enter or pass through, optionally perpendicular to the hook axis. In an exemplary embodiment of the invention, the tabs are formed on the eye and the apertures are formed on the hook. In an exemplary embodiment of the invention, the eye is pre-stressed to distort it such that the tab interlocks with the aperture. However, the hook is aligned to allow such interlocking only when the connector is deployed, for example, by retracting the hook. In some exemplary embodiments of the invention, the tab is formed on a part of the eye that, when distorted, does not change the external shape of the eye. Alternatively, the tab is formed on an external portion of the eye, which distorts to provide interlocking.

In an exemplary embodiment of the invention, the eyes segments are not interconnected and the hooks segments are interconnect prior to tearing.

An aspect of some embodiments of the invention relates to a tearing mechanism for a hook and eye based connector in which the tearing mechanism is independent of a locking mechanism used to lock or interlock the hook and eye, which locking is applied prior to tearing.

An aspect of some embodiments of the invention relates to an anastomotic connector for side to end connection, in which the lips of the side vessel are minimally distorted during and after the connection is made. In an exemplary embodiment of the invention, the lips of the end vessel are everted less than 180°, for example about 9020 .

An aspect of some embodiments of the invention relates to a stitching connector for an end-to-side or end-to-end anastomosis, which transfixes the end vessel lip twice. In an exemplary embodiment of the invention, the first transfixing is during eversion while mounting the connector on the end vessel. the second eversion is during deployment. In an exemplary embodiment of the invention, the first transfixing is radially outside of the second transfixing.

There is thus provided in accordance with an exemplary embodiment of the invention, a transfixing assistance device for transfixing a blood vessel on one or more hooks of an anastomotic connector, said transfixing device comprising:
a handle defining a longitudinal axis; and
an extension projecting from said handle comprising an orifice at its end, said extension being offset at an angle to said longitudinal axis so that, when said handle is rotated during said transfixing, said tip circumducts at a radius corresponding to its offset angle to said longitudinal axis, said radius suitable for everting a graft over a spiked anastomotic connector. Optionally, said orifice is adapted to transfix blood vessel tissue on said one or more connection hooks without damaging said-tissue.

In an exemplary embodiment of the invention, said device comprises two or more opposable extension projection from said handle, arranged to function as a forceps.

In an exemplary embodiment of the invention, said orifice comprises a closed aperture.

There is also provided in accordance with an exemplary embodiment of the invention, a transfixing assistance device for transfixing a blood vessel on one or more hooks of an anastomotic connector, said transfixing device comprising:
two elongate members attached at a base thereof;
an orifice adapted to transfix blood vessel tissue on said one or more connection hooks without damaging said tissue, defined on at least one of said members.

There is also provided in accordance with an exemplary embodiment of the invention, a method of guiding hooks of an anastomotic connector into an aperture of a blood vessel, comprising:
surrounding said hooks with a mechanical element that compresses them towards each other;
inserting said compressed hooks into an aperture of a blood vessel; and
releasing said hooks. Optionally, surrounding comprises protecting said hooks from tissue adjacent said vessel. Alternatively or additionally, surrounding comprises inserting said hooks into a guide. Alternatively or additionally; surrounding comprises inserting said hooks into a hook arranger. Alternatively or additionally, surrounding comprises inserting said hooks into a wire loop.

There is also provided in accordance with an exemplary embodiment of the invention, a hook arranging device for arranging a plurality of anastomotic hooks projecting from an anastomotic connector, said hook arranging device comprising:
one or more arranging plates adapted to arrange a plurality of hooks of an anastomotic connector; and
a coupler adapted for coupling said at least one plate to an anastomotic connector delivery system. Optionally, said one or more plates are removably connected to said delivery system.

In an exemplary embodiment of the invention, said one or more plates comprise two edges that presses said plurality of hooks between them. Alternatively or additionally, said one or more plates comprise one or more spacers that space two or more of said plurality of hooks.

There is also provided in accordance with an exemplary embodiment of the invention, a guide for guiding a plurality of anastomotic hooks into a blood vessel, said guide comprising:
a guide tip adapted to be placed through an aperture in a blood vessel, said tip being further adapted to guide a plurality of hooks into said blood vessel without contacting said aperture; and
at least one guide wall attached to said tip, said at least one wall being adapted to guide said plurality of hooks toward said tip while protecting said hooks from contacting tissue in proximity to said aperture. Optionally, said tip is adapted to protect said hooks from contacting the edges of said aperture during said guiding.

In an exemplary embodiment of the invention, said tip comprises a blunt end. Alternatively, said tip comprises a sharp end adapted to form said aperture.

In an exemplary embodiment of the invention, said at least one wall comprises two or more walls. Optionally, said two or more walls are connected to each other.

In an exemplary embodiment of the invention, said guide is adapted to remove from said blood vessel following guiding said plurality of anastomotic hooks.

In an exemplary embodiment of the invention, said guide is rotatably mounted on an anastomotic connector delivery system, such that said hooks selectively enter said guide by said rotation.

There is also provided in accordance with an exemplary embodiment of the invention, a retractable hook grasper for retractably grasping a plurality of anastomotic connector hooks, comprising:
a handle;
at least one grasping wire projecting from said handle, adapted to grasp and compress a plurality of anastomotic connector hooks; and
a grasping wire controller that controls extension of said extension wire in relation to said handle, such that at one extension, the grasping wire receives uncompressed hooks, at a 30 second extension the grasping wire compresses the hooks and at a third, further extension, the hooks are released. Optionally, said adaptation to grasp a plurality of hooks comprises a curvature adapted for partially encircling a plurality of said hooks. Alternatively, said adaptation to grasp a plurality of hooks comprises a form of a loop. Optionally, said wire defines at least one breakaway area on said wire that breaks when said wire is pulled away taunt.

In an exemplary embodiment of the invention, said extensions comprises progressive retraction positions of said wire.

In an exemplary embodiment of the invention, said hook grasper on a connector delivery system.

In an exemplary embodiment of the invention, said grasper comprises a tube into which said wire is retracted between the extension position.

There is also provided in accordance with an exemplary embodiment of the invention, a blood vessel cutter for cutting an aperture in a blood vessel, comprising:
a handle having a longitudinal axis;
a horn-shaped cutting edge connect to said handle, said cutting edge describing an arc around said longitudinal axis upon rotation of said handle around said longitudinal axis;
whereby, said cutting edge is adapted to cut an aperture in a blood vessel when held proximate to said vessel during said rotation.

There is also provided in accordance with an exemplary embodiment of the invention, an anastomotic delivery system for delivering an anastomotic connector into a blood vessel and tearing one or more extensions off of said connector, said system comprising:
a puller which is coupled to said extensions;
a manual input operative to retract said puller;
a loaded spring, coupled to said puller;
a selectable interlock which selectively prevents a release of said spring; and

an interlock release, coupled to said manual input, and operative to release said interlock depending on a retraction of said puller, wherein releasing said interlock releases said spring to tear said extensions. Optionally, said system comprises a shock absorber to reduce a delivery of shock from said spring to a housing of said system, when said spring is released.

There is also provided in accordance with an exemplary embodiment of the invention, an anastomotic connector rotating device, comprising:
a handle; and
an anastomotic connector holder rotatably attached to said handle. Optionally, said rotator comprises one or more rotational extent rests.

In an exemplary embodiment of the invention, said device comprises a rotational extent indicator.

There is also provided in accordance with an exemplary embodiment of the invention, an two part anastomosis delivery system, comprising:
a handle section, adapted to apply force sufficient to deploy an anastomotic connector, through a coupling thereof; and
a capsule adapted to be removably mounted on said handle and to apply said force through said coupling to a connector mounted on said capsule. Optionally, said capsule is rotatably mounted on said handle section. Alternatively or additionally, said capsule includes a hook retractor operative to manually retract and extend hooks of said connector. Optionally, said capsule-comprises two axial sections which rotate one relative to the other to effect said extension and retraction. Alternatively or additionally, said capsule comprises an axially moving pin which effects said extension and retraction. Alternatively or additionally, said capsule comprises a pin which rotates around an axis of said capsule to effect said extension and retraction. Alternatively or additionally, said handle section prevents access to said hook retractor, when said capsule is mounted on said handle. Alternatively or additionally, said capsule is adapted to not mount on said handle section if said hooks are not in a pre-defined axial position.

In an exemplary embodiment of the invention, said capsule comprises a stop which restricts axial motion of said hooks.

There is also provided in accordance with an exemplary embodiment of the invention, an anastomotic connector for attaching two blood vessels comprising:
a plurality of eye segments, each defining a channel and each including a part of an interlock mechanism on said channel;
a plurality of hook segments, each defining a tissue holding area, each adapted to pass through said channel and including a second part of said interlock mechanism, wherein, said interlock mechanism engages for a hook and an eye segment when said hook segment is retracted back into said eye segment enough to attach two layers of vascular tissue between said eye segment and said hook segment. Optionally, said hook segment comprises a curved hook tip having a sharpened tissue penetrating tip at its end. Optionally, said tip is generally aligned with a center of said eye segment. Optionally, said eye segment defines an aperture aligned with said tip. Optionally, said eye segment comprises at least one flap in said aperture, to reduce tissue ingress into said aperture.

In an exemplary embodiment of the invention, said eye segment comprises a body of a closed ring.

In an exemplary embodiment of the invention, said eye segment comprises a body which is open at at least one point of its circumference.

In an exemplary embodiment of the invention, said interlocking mechanism is stiff enough and strong enough to hold said hook segment while it is being torn off an extension thereof, by pulling on the extension.

In an exemplary embodiment of the invention, said hook segment includes an extension which is torn off said hook segment during deployment by pulling, said hook segment defining a rest stop where said hook segment is held during said pulling. Optionally, said extension defines a slot terminating at said rest stop.

In an exemplary embodiment of the invention, said interlocking mechanism is substantially all on a plane of said eye segment, once interlocked.

In an exemplary embodiment of the invention, said interlocking mechanism comprises at least one tab that is perpendicular to an axis of said hook segment, at said channel.

In an exemplary embodiment of the invention, said tab enters a matching aperture formed in said hook segment. Alternatively or additionally, said tab transfixes a matching aperture formed in said hook segment. Alternatively or additionally, said tab transfixes a matching open slot formed in said hook segment.

In an exemplary embodiment of the invention, the connector comprises at least one spring element which approximates said channel and said tab. Optionally, said tab is mounted on said spring element. Alternatively said tab is not mounted on said spring element. Optionally, said spring element urges said hook element against said tab.

In an exemplary embodiment of the invention, said spring element is formed of an outer portion of said eye segment.

In an exemplary embodiment of the invention, said spring element is attached to said eye segment near said channel.

In an exemplary embodiment of the invention, said spring element is attached to said eye segment far from said channel.

In an exemplary. embodiment of the invention, said eye segment includes a support bar on which said spring element is attached.

In an exemplary embodiment of the invention, said at least one tab comprises only a single tab.

In an exemplary embodiment of the invention, said at least one tab comprises at least two tabs.

In an exemplary embodiment of the invention, said hook element includes an extension which is torn off during deployment, said extension defining an alternative aperture for locking said tab spaced from said tissue holding area.

In an exemplary embodiment of the invention, said eye segments are interconnected after deployment.

In an exemplary embodiment of the invention, said eye segments are not interconnected after deployment.

There is also provided in accordance with an exemplary embodiment of the invention, an anastomotic connection clip element, comprising:
a base;
at least one stitching spike, attached to said base and having a sharp end adapted to penetrate vascular tissue; and
at least one top spike, attached to said base,
wherein said stitching spike and said top spike diverge in opposite directions near said base curve back towards each other away from said base. Optionally, said spikes comprises at least one spike of one type and two spikes of the other type, interleaved. Optionally, said top spike is curved to conform to a blood vessel curvature. Alternatively or additionally, said element is coupled to a plurality of clip elements, to form a connector.

### BRIEF DESCRIPTION OF THE FIGURES

Non-limiting embodiments of the invention will be described with reference to the following description of exemplary embodiments, in conjunction with the figures. The Figures. are generally not shown to scale and any sizes are only meant to be exemplary and not necessarily limiting. In the figures, identical structures, elements or parts that appear in more than one figure are preferably labeled with a same or similar number in all the figures in which they appear, in which:

FIGS. 1A-1G are schematic views of an anastomotic connector and its deployment as described above, in accordance with a previous publication;

FIG. 2A is an isometric view of an anastomotic delivery system, in accordance with an exemplary embodiment of the invention;

FIG. 2B is a close-up view of a portion of the delivery system of FIG. 2A, in accordance with an exemplary embodiment of the invention;

FIG. 3 is a detail of a rotation extent controlling element, in accordance with an exemplary embodiment of the invention;

FIG. 4 is a partially exploded view of a portion of the delivery system of FIG. 2A, incorporating the coupler of FIG. 3, in accordance with an exemplary embodiment of the invention;

FIGS. 5A-5B are isometric views of a transfixing assistance device, in accordance with an exemplary embodiment of the invention;

FIGS. 5C-5D are isometric views of a transfixing assistance forceps, in accordance with an exemplary embodiment of the invention;

FIGS. 6A-6C demonstrate the operation of the transfixing assistance device of FIGS. 5A-5B, in accordance with an exemplary embodiment of the invention;

FIGS. 6D-6F demonstrate the operation of the transfixing assistance device of FIGS. 5C-5D, in accordance with an exemplary embodiment of the invention;

FIG. 7 is an isometric view of a hook arranging device mounted on a portion of an anastomotic delivery system, in accordance with an exemplary embodiment of the invention;

FIGS. 8A-8C are schematic views demonstrating the operation of an anastomotic connector guide, in accordance with an exemplary embodiment of the invention;

FIGS. 9A-9B demonstrate the operation of an alternative embodiment of the guide of FIGS. 8A-8C, in accordance with an exemplary embodiment of the invention;

FIG. 10 is an alternative embodiment of the guide of FIGS. 8A-8C, in accordance with an exemplary embodiment of the invention;

FIGS. 11A-C demonstrate the operation of a hook stabilizing device, in accordance with an exemplary embodiment of the invention;

FIG. 12 is an isometric view of a blood vessel cutter, in accordance with an exemplary embodiment of the invention;

FIG. 13 is a cut-away view of a spring-loaded anastomosis connector delivery system, in accordance with an exemplary embodiment of the invention;

FIG. 14 is a cut-away view of breakable spike receptacle, in accordance with an exemplary embodiment of the invention;

FIG. 15 is an isometric view of the delivery system of FIG. 13 and the spike receptacle of FIG. 14 operating together, in accordance with an exemplary embodiment of the invention;

FIGS. 16A-16E illustrate an interlocking anastomotic connector portion, in accordance with an exemplary embodiment of the invention;

FIGS. 17A-171 illustrate several variations of hook segment 1602, in accordance with exemplary embodiments of the invention;

FIGS. 18A-18G illustrate various locking mechanisms, all of which are optionally in the plane of the eye segment, in accordance with an exemplary embodiment of the invention;

FIGS. 19A-19E show eye segments in accordance with an exemplary embodiment of the invention;

FIGS. 20A-20B show a deployment mechanism for the connector portion of FIGS. 16A-16D, in accordance with an exemplary embodiment of the invention;

FIGS. 21A-21D illustrate a spike clip in accordance with an exemplary embodiment of the invention; and

FIGS. 22A-22D illustrate a spiked clip which stitches two blood vessel lips together by transfixing a first vessel, transfixing a second vessel and then optionally transfixing the first again, in accordance with an exemplary embodiment of the invention.

### DETAILED DESCRIPTION OF EXEMPLARY EMBODIMENTS

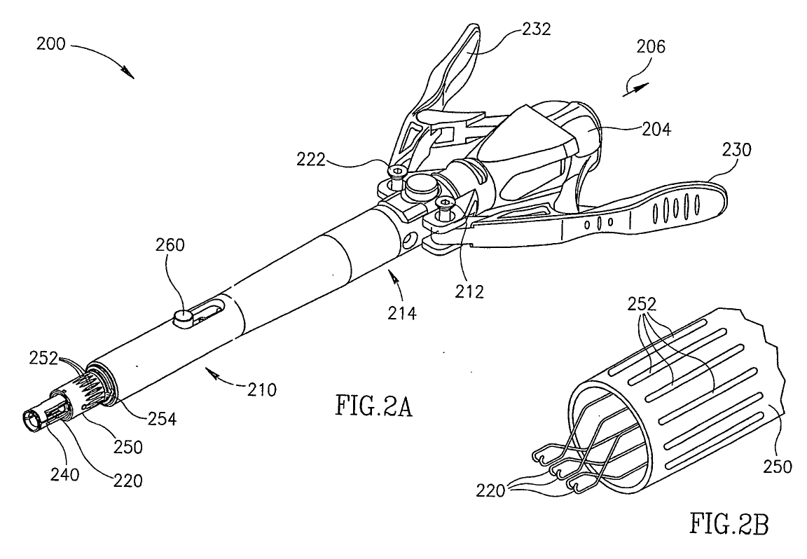

FIG. 2A is an isometric view of an anastomotic delivery system 200 in accordance with an exemplary embodiment of the invention. System 200 comprises a body 214. A pair of levers 230 and 232 is attached to rotation pins 222 and 212 respectively. Pressing together levers 232 and 230 causes a base 204 to move in a direction 206, as they are pressed toward each other, thereby causing a plurality of hooks 220 of a connector to retract partially into body 214, and then optionally tear off of system 200, for example as described in PCT publication WO 01/70090.

In an exemplary embodiment of the invention, the connector is mounted on a rotation sleeve 250, which allows the connector to be rotated relative to body 214. FIG. 2B is a close-up view of a rotation sleeve 250 of delivery system 200, shown in FIG. 2A, with a protector overtube 240 removed, in accordance with an exemplary embodiment of the invention. Rotation sleeve 250 has been rotated so that hooks 220 are on a horizontal plane. Optionally, the tips of hooks 220 are arranged in a straight line for example corresponding to a straight aperture in a blood vessel, for example as described below.

In an exemplary embodiment, protector overtube 240 surrounds hooks 220 to protect delicate hooks 220 from being damaged during placement in a host vessel. The tips of the hooks are optionally covered by a cap (not shown) that mounts over overtube 240. A graft (not shown) enters through a side opening in tube 240 and is everted over hooks 220.

Rotation sleeve 250 allows rotation of handle 210 and/or levers 230 and/or 232 in relation to anastomotic connector hooks 220 (FIG. 2B) so that handles 210 and/or levers 230 and/or 232 do not inadvertently brush against biological tissue. Proper protection and/or alignment of device 200 is useful when performing an anastomosis to allow ease of operation by an operator, without damaging surrounding tissue.

As described for example in WO 00/56226, the disclosure of which is incorporated herein by reference, overtube 240 may be splittable lengthwise, for example using a pin 260, so that overtube 240 and/or rotation sleeve 250 may be split open and removed following anastomosis of a vessel using hooks 220.

Additionally or alternatively, pin 260 may have other functions, for example, to control longitudinal movement of spikes 228 and/or hooks 220 in relation to rotation sleeve 250. Pin 260, for example, pulled in direction 206 retracts hooks 220 into rotation sleeve 250, and advanced to exposing hooks 220, for example during an anastomosis procedure. Exposed hooks 220 are thereby free to enter an aperture in a blood vessel and/or connect the grafted vessel to the host vessel. In an exemplary embodiment of the invention, the retraction is practiced during eversion of the graft. In an exemplary embodiment of the invention, retracted hooks are shorter and therefor stiffer, which may be important during eversion if the hooks are too flexible.

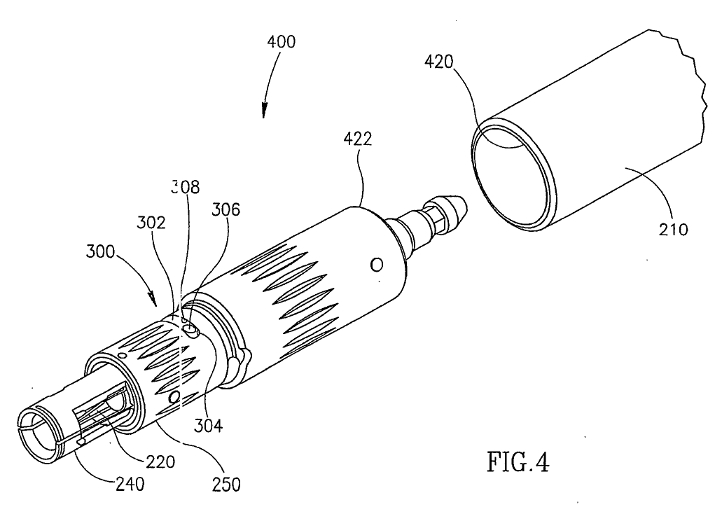

In an exemplary embodiment, as rotation sleeve 250 is rotated in relation to handle 210, audible clicking noises are provided (for example generated by the hexagonal coupling shown in FIG. 4) to indicate that it is in motion and/or as a side effect of a position locking mechanism. Additionally or alternatively, the clicking noise indicates the axial position of handles 230 and/or 232. Alternatively or additionally, rotation of rotation sleeve 250 provides visual indication of rotation and/or extent of rotation.

In an exemplary embodiment of the invention, rotation sleeve 250 is part of a separable capsule (e.g., as shown in FIG. 4). In this case, some or all of the rotation may be performed before attaching the capsule to the rest of the delivery system. Optionally, in use, the tip of the delivery system is held in place (e.g., against a target vessel) and the body is rotated.

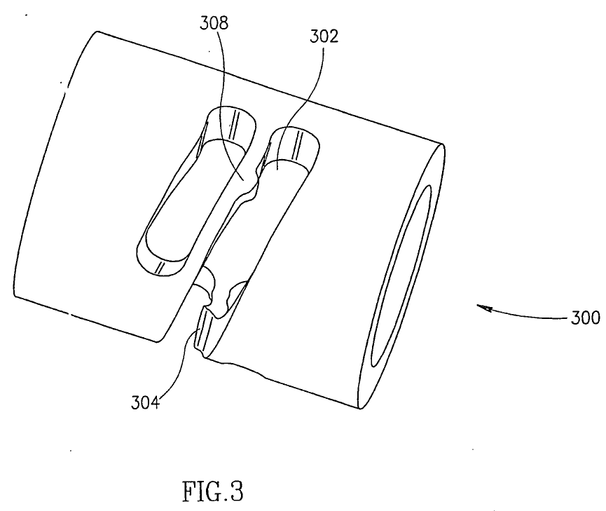

FIG. 3 is a detail of a coupler 300 with an extent channel 302 comprising an extent rest 308 and an extent stop 304, in accordance with an exemplary embodiment of the invention. this coupler, internal to system 200, is used to control the relative rotation of two parts of system 200, for example relative rotation of a capsule as described in FIG. 4, below, in which rotation is used for axial motion of hooks 220, retraction and/or extension. In an exemplary embodiment of the invention, the extent stops are used to prevent over retraction and/or over extension of hooks 220.

FIG. 4 is an exploded view of a portion of delivery system 200 of FIG. 2A, incorporating coupler 300 of FIG. 3, in accordance with an exemplary embodiment of the invention. A separate capsule 400 is shown. In an exemplary embodiment of the invention, the eversion is performed on capsule 400, for example being held by hand or on a suitable mount. Then the capsule is mounted onto system 200 and used. Optionally, an extension rod may be attached between the capsule and the handle. The coupling between the capsule and the delivery system may be, for example solid hexagonal as shown. Alternatively or additionally, it may be hollow, for example to allow a tool, such as a punch mechanism, or a power source or a control mechanism, to pass through it.

In an exemplary embodiment of the invention, capsule 400 includes a mechanism for selective retraction and extension of hooks 220. Not all connectors will require such a mechanism but it may be useful for connectors with very flexible and/or long hooks, to simplify eversion. Alternatively or additionally, this may be useful for matching the projecting length of the hooks with the diameter of the target vessel, for example to ensure that the hooks enter and/or to prevent them contacting the far wall of the target vessel.

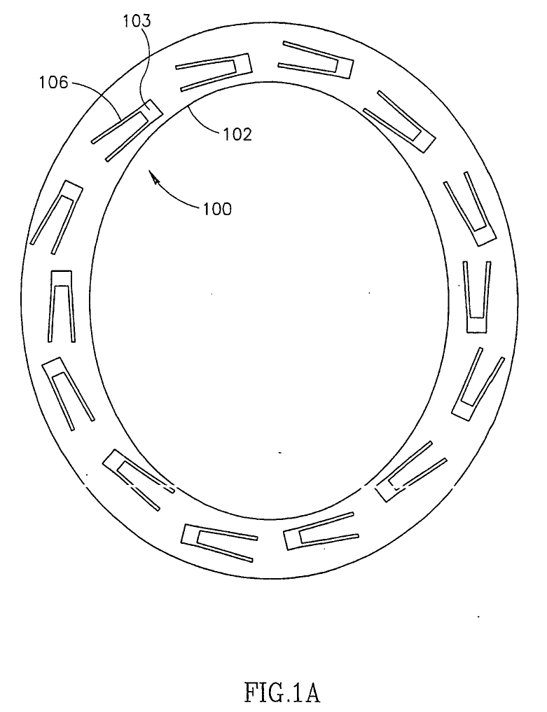

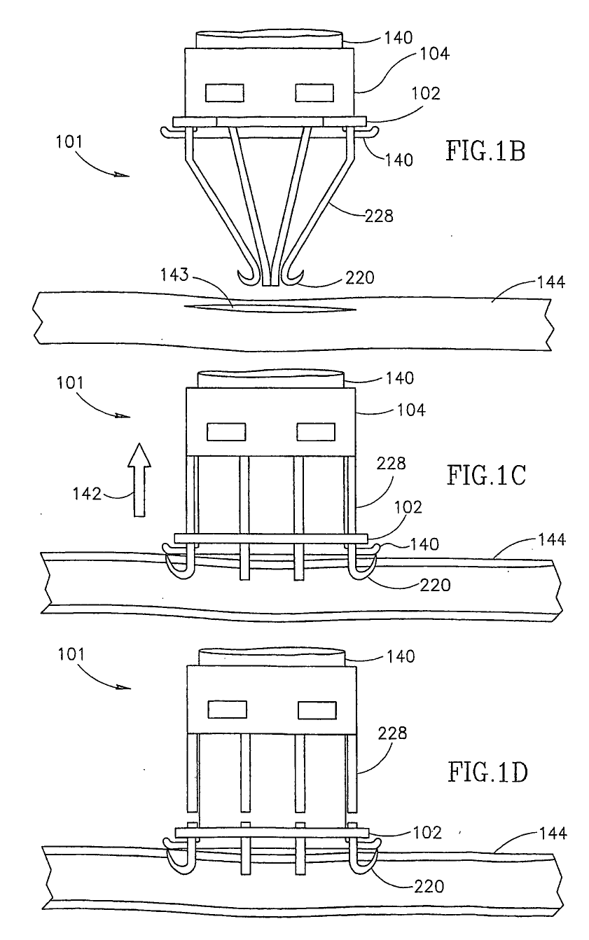

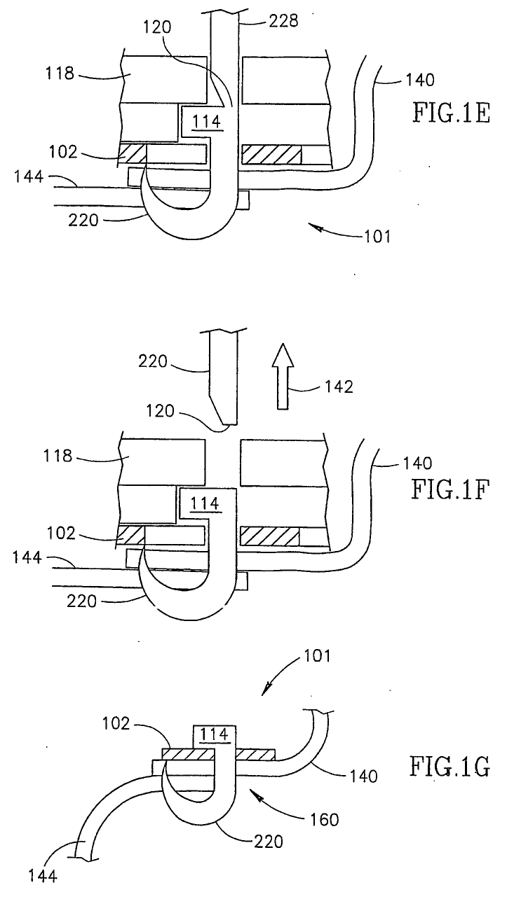

Optionally, hooks 220 are pre-stressed to form a cone shape. In an exemplary embodiment of the invention, when the hooks are retracted, the cone shape is opened, for example as shown in FIG. 1.

Stop 304 of extent channel 302 prevents further rotational movement and locks the position of hooks 220 as a pin (not shown) presses against stop 304.

In an exemplary embodiment of the invention, one or more projections, for example a sleeve, project out of capsule 400 in the direction of system 200 and prevent interlocking of capsule 400 and system 200 if hooks 220 are not in a desirable retracted or extended position. The axial position of the projection is controlled by the rotation mechanism, for example the coupling of FIG. 3. Omier mechanisms which prevent interlocking may be used instead.

In an exemplary embodiment of the invention, when handle. 210 is assembled with capsule 400, handle 210 covers capsule 400, leaving, for example, extent channel 302 projecting forward of edge 420. With capsule 400 covered by handle 210, an operator is prevented from inadvertently rotating capsule 400 in relation to handle 210 in a mistaken attempt to advance/retract hooks 220. Alternatively or additionally, capsule 400 may have an oval cross-section, which prevents rotation. Alternatively or additionally, where a pin is used for moving the hooks, this pin is covered by the delivery system, Alternatively to an axial moving pin or a rotating sleeve, a rotating pin may be used, in which only a pin that extends through a slot in capsule 400 rotates around the axis of capsule 400 and not an entire sleeve 250.

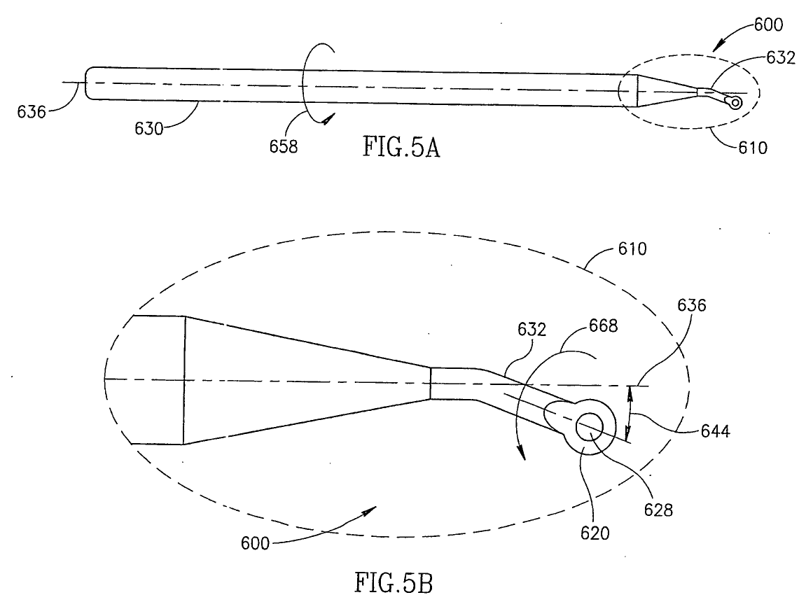

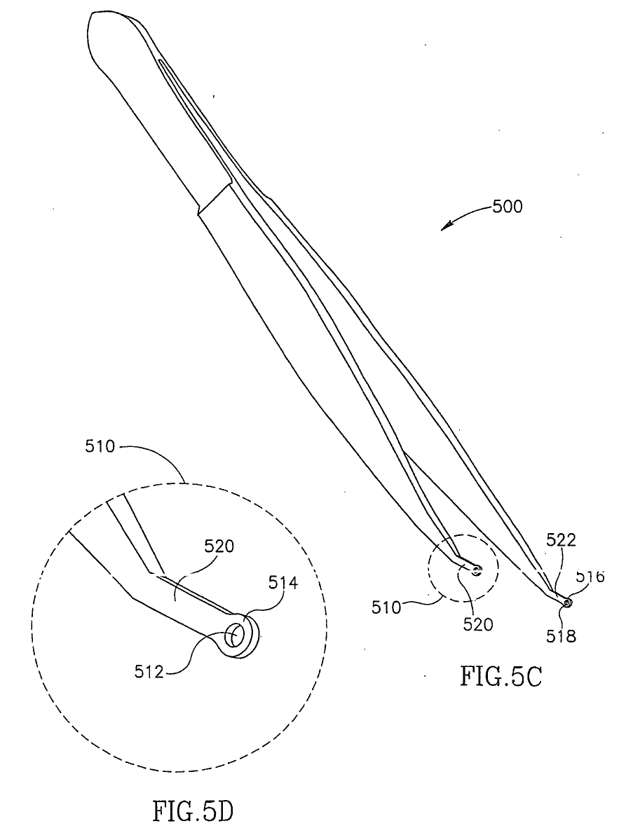

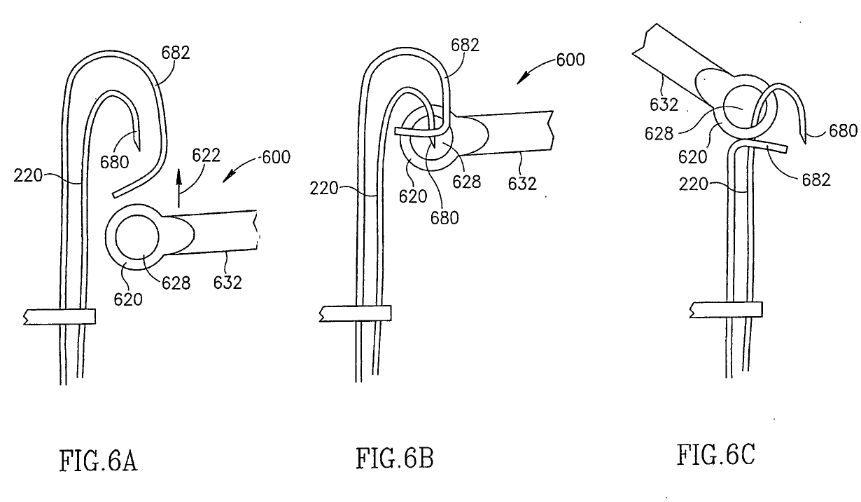

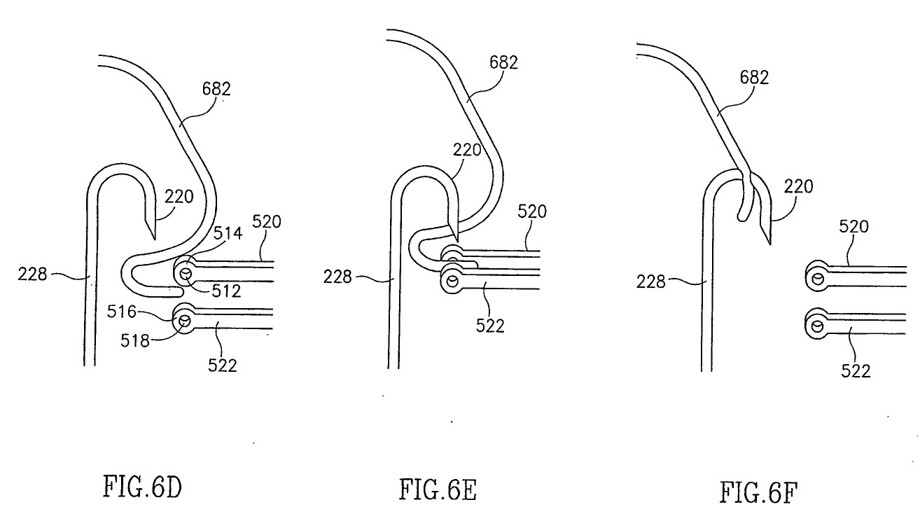

FIGS. 5A-5B are side views of a transfixing assistance device 600, in accordance with an exemplary embodiment of the invention, for transfixing a blood vessel 682 (FIG. 6C) on one or more anastomotic connector hooks 220 (FIG. 6C). FIGS. 6A-6C show how device 600 is used for transfixing, in accordance with on embodiment of the invention. In an exemplary embodiment, transfixing assistance device 600 comprises a handle 630 defining a longitudinal axis 636 with an extension 632 at an offset angle 644 from longitudinal axis 636. In an exemplary embodiment, handle 630 is rotated in direction 658 so that extension 632 circumducts 668 around longitudinal axis 636, meaning that its tip describes a circle. In an exemplary embodiment, transfixing of a blood vessel on hook 220 is accomplished without damage to either hook 220 or the biologic tissue, due to its design incorporating circumduction 668 of extension 632.

FIGS. 6A-6C demonstrate the operation of transfixing assistance device 600 of FIGS. 5A-5B, in accordance with an exemplary embodiment of the invention. Extension 632 has an orifice 628 along a tip 620 that is large enough to accommodate at least a part of a blood vessel wall 682 as it is draped over hook 220 without damage during said transfixing. Possibly, not an entire wall can be accommodated, so that the vessel wall will not fold over hook 220. However, orifice 628 is generally larger than hook 220. For example, the orifice may be 0.65 mm in diameter and the hook be have a cross-section of 0.15 mm by 0.45 mm.

Hook 220 has a curved head 680 upon which a vessel wall 682 is transfixed. In an exemplary embodiment, extension 632 circumducts 668 around longitudinal axis 636 so that tip 620 follows the trajectory of curved head 680.

In FIG. 6A, orifice 628 is aligned with vessel wall 682 as it overlaps curved head 680. The vessel wall has been everted and is optionally caught (not shown for clarity) on hook 220. However, vessel wall 682 may not be not transfixed. In FIG. 6B, tip 620 has been placed against vessel wall 682 so that vessel wall 682 is impaled on curved head 680. Handle 630 is now rotated by the operator, thereby circumducting 668 extension 632 so that orifice 628 follows the curvature of curved head 680. In FIG. 6C, circumduction 686 of extension 632 has been completed and vessel wall 682 has been successfully transfixed and pulled over curved head 680 of hook 220.

In various exemplary embodiments, orifice 628 may have a variety of shapes: oval, rectangular or triangular, and/or a variety of orifice sizes, to accommodate a variety of vessel wall 682 thicknesses and/or tissue configurations. In an exemplary embodiment, tip 620 incorporates soft materials and/or coatings that are soft, absorb shock and/or move easily over wet, moving tissue. In another example, an open orifice, for example a slot, is used. The size of the aperture and the direction of offset 632 are exaggerated, for clarity.

FIGS. 5C and 5D show an isometric view of an alternative transfixing assistance device 500 comprising a transfixing forceps with two legs 520 and 522, one or both legs 520 and 522 ending in a transfixing extension 514 and/or 518. Extension 514 and/or 518, for example comprise orifices 512 and 516 respectively, adapted for encouraging movement of a blood vessel 682 wall on hook 220. One potential advantage of the forceps device is that a same device (e.g., the forceps) may be used to evert the vessel and then transfix it over the hooks.

FIGS. 6D-6F demonstrate the operation of transfixing assistance forceps 500 of FIGS. 5C-5D. In FIG. 6D, transfixing forceps 500 (shown open, for clarity) grasp a wall of blood vessel 682, and evert it onto connector hook 220. FIG. 6E shows that forceps 500 may be used to catch vessel wall 682 onto hook 220. Now, the process as shown in FIGS. 6B-6C may be performed. FIG. 6F shows an intermediate stage where the vessel wall is not completely pushed back over hook 220.

Even if the hooks are arranged to have their tips in the shape of a cone (possibly a flattened cone), it may be difficult to fit the hooks into an incision, which may be a linear or curved cut, rather than a hole. Also, if the target vessel is collapsed, it may be difficult to manipulate the hooks.

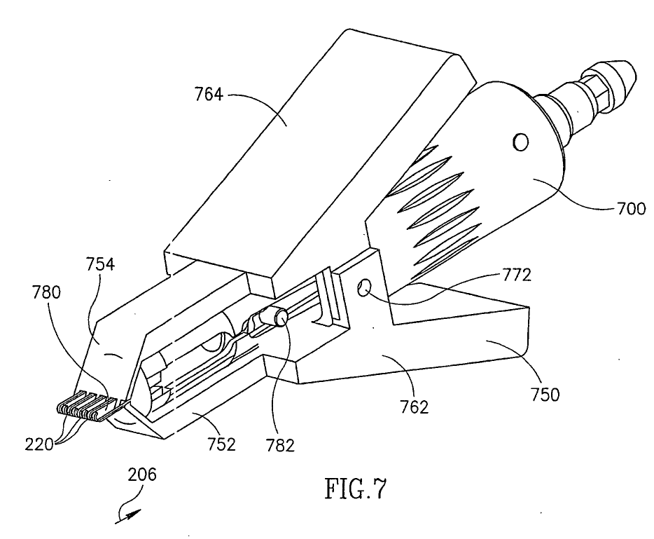

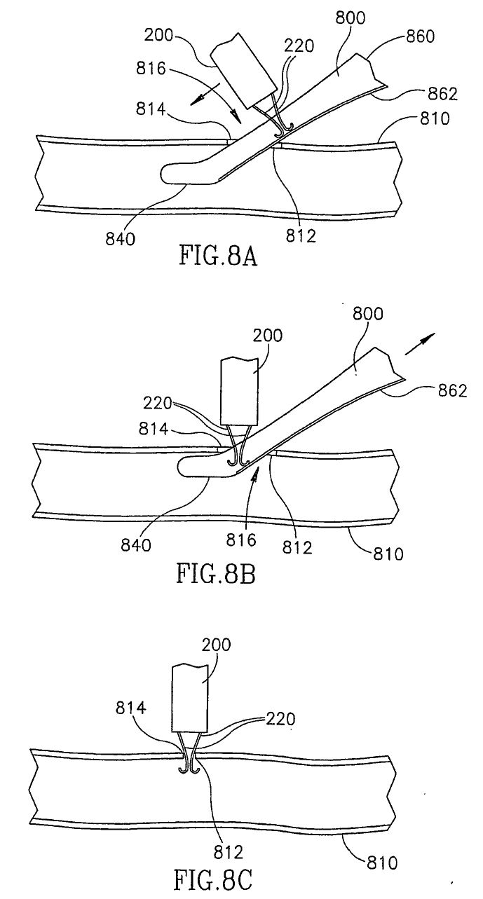

FIG. 7 is an isometric view of an arranger 750 for hooks 220, mounted on an anastomotic delivery system capsule 700, in accordance with an exemplary embodiment of the invention. Hook arranger 750 comprises one or more plates 754 and/or 752 that keep hooks 220 in a specific arrangement in relation to, for example, aperture 812 in blood vessel 816 (FIG.8C). Alternatively or additionally, arranger 750 may be used for opening the incision, while preventing the hooks from inadvertently engaging tissue.

Additionally or alternatively, hook arranger 750 presses hooks 220 together so that there are no projecting sharp edges that can brush against vessel 810 and bend. This arrangement of hooks 220 is shown in FIG. 2B. Note that when hooks 220 are compressed together from their natural circular position alternate hooks 220 point in opposite directions. In this fashion, the points of hooks 220 are prevented from inadvertently hooking into biologic tissue and being damage.

In an exemplary embodiment, arranger 750 maintains hooks 220 in a straight linear formation. Additionally or alternatively, arranger 750 maintains hooks 220 in a curvilinear formation so that hooks 220 conform to a curvilinear aperture 812 in blood vessel 810 (not shown), in which the tips optionally protect each other. Alternatively or additionally, plates 754 define guiding slots, one for each hook, possibly covering the hooks from the outside. In an exemplary embodiment a handle 764 attached to plate 754, and a handle 762 attached to plate 752, cause plates 752 and 754 to move away from each other when handles 762 and 764 are pressed toward each other, thereby releasing the hooks.

Alternatively or additionally plates 752 and/or 754 comprise one or more spacers 780 that, for example, press against a hook 220 and/or pass between two or more hooks 220. In an exemplary embodiment, spacers 780 are removed from hooks 220 as handles 762 and 764 are pressed toward each other as they pivot on a pin 772.

In an exemplary embodiment, a safety pin 782 is provided that splits cone 250 and/or other parts of capsule 700 following an anastomotic connection, as mentioned above.

FIGS. 8A-8C are schematic views demonstrating the operation of a guide 800 for protecting a plurality of anastomotic hooks 220 projecting from anastomotic delivery system 200 during movement of said hooks 220 toward and/or into a blood vessel 810, in accordance with an exemplary embodiment of the invention.

Guide 800 comprises at least one wall 860 that guide hooks 220 as anastomotic delivery system 200 is moved toward blood vessel 810, thereby preventing hooks 220 from coming in contact with blood vessel 810 and/or biologic structures around blood vessel 810. Such contact could cause, for example, hooks 220 to become bent so they improperly align with aperture 816 in blood vessel 810 and, provide a less than optimal anastomosis with blood vessel 810.

In an exemplary embodiment, guide 800 comprises a guide tip 840 designed to enter blood vessel 810 between edges 812 and 814 (into aperture 816) without damaging edges 812, 814. Optionally, guide tip 840 prevents damage to hooks 220 during placement in blood vessel 810.

In an exemplary embodiment, guide 800 and/or tip 840 comprise a curved tube, with a guidance slot defined along the inside of the curve. In an exemplary embodiment, walls 860 and/or 862 are spaced (e.g., the width of the slot) so that hooks 220 move easily through guide 800 without contacting blood vessel 810 and/or surrounding biologic tissue. Alternatively or additionally, walls 860 and/or 862 spaced from each other to facilitate easy movement of hooks 220 into blood vessel 810. In an exemplary embodiment, two or more walls 860 and 862 comprise one or more flared openings, for example at one or both ends, to facilitate entry and/or exit of hooks 220 from guide 800. The spacing may or may not be constant along the length of the guide, for example, it may become progressively narrower, independent of any flaring.

In an exemplary embodiment, guide tip 840 is configured so that following its placement through aperture 816 and setting hooks 220 in blood vessel 810, guide 800 is easily removed from aperture 816 while hooks 220 remain in place (FIG. 8C). For example, tip 840 is made substantially narrower than the incision and/or is flat enough to fit between the hooks and the delivery system.

FIG. 8A shows the guide inserted into an aperture 816 of a blood vessel and hooks 220 traveling along it.

FIG. 8B shows the hooks 220 having reached into the aperture.

FIG. 8C shows the effect of removing the guide: the hooks are released and the incision can possibly close over the hooks.

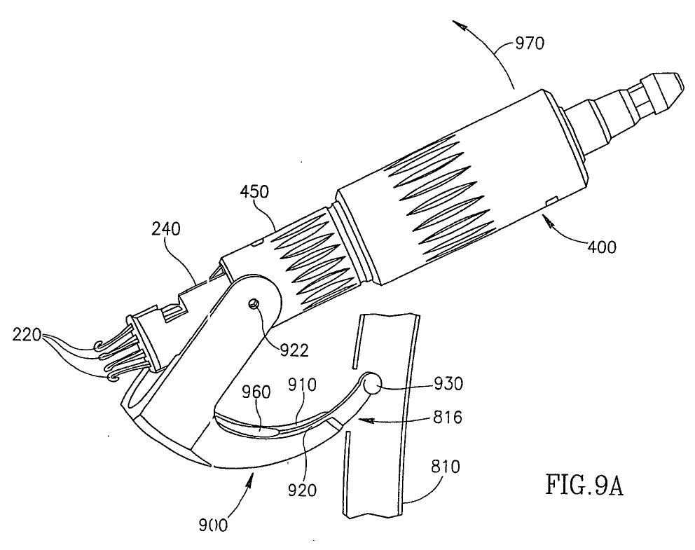

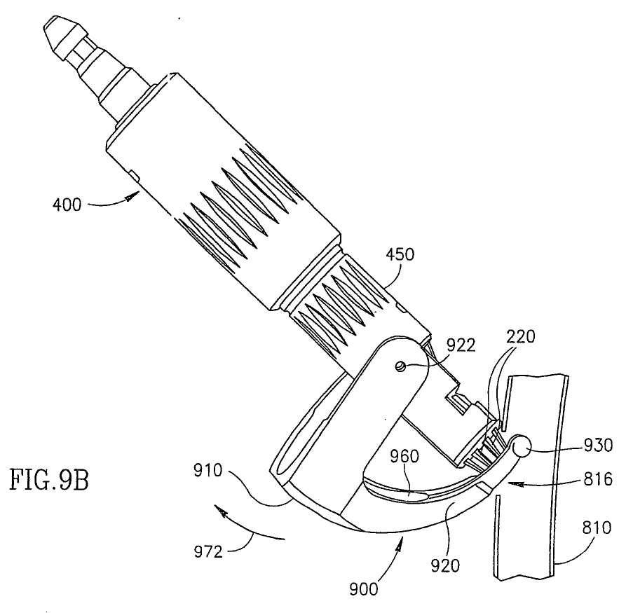

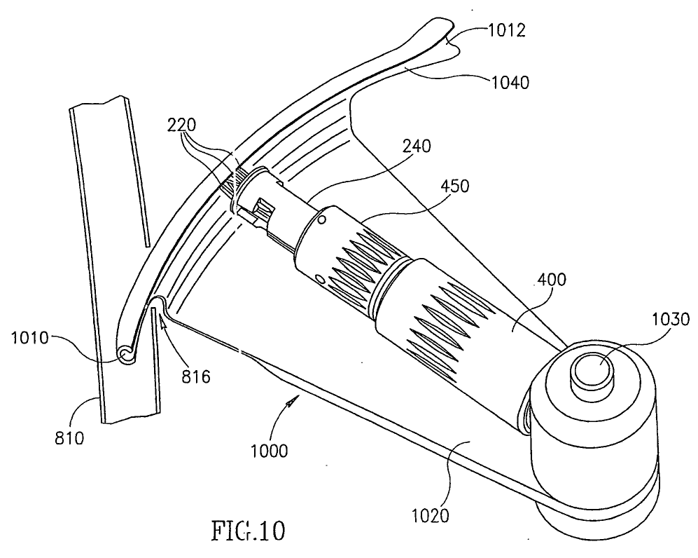

FIGS. 9A-9B demonstrate the operation of a guide 900, mounted on an extension 450 of capsule 400, in accordance with an exemplary embodiment of the invention. In an exemplary embodiment, guide 900 comprises a channel 960 and a tip 930 designed to enter aperture 816 in blood vessel 810. In an exemplary embodiment, a first wall 910 and/or a second wall 920 of guide 900, prevent contact between hooks 220 and surrounding biologic tissue, for example walls of blood vessel 810.

In an exemplary embodiment, guide 900 is pivotally attached to extension 450 with a pivot pin 922 so that with guide 900 in blood vessel 810, capsule 400 is rotated in a direction 970 bringing hooks 220 into blood vessel 810.

In an exemplary embodiment, after hooks 220 are in place in blood vessel 810, guide 900 is pivoted in a direction 972 (FIG. 9B) so that it exits blood vessel 810, leaving hooks 220 in place.

While an arc shaped guide is shown, other shapes may be used. For example, a spiral shaped guide, with one, fewer or more turns may be useful for narrow areas. Instead of merely rotating the delivery system relative to the guide, the tip of the delivery system may move in the shape of a spiral, to follow the guide shape. A gimbaling hinge may be used in stead of a planar pivot hinge shown.

FIG. 10 is an alternative embodiment of a guide 1000 mounted on (or integral with) capsule 400 with a pivot pin 1030, in accordance with an exemplary embodiment of the invention. In an exemplary embodiment, guide 1000 comprises a wall 1020 that prevents capsule 400 from contacting biologic tissue, for example, during implantation of hooks 220 in blood vessel 810. Wall 1020, for example, of guide 1000, may be held by an operator to aid in guiding guide 1000 toward aperture 812 of vessel 810.

In an exemplary embodiment, guide 1000 comprises a channel 1040 with a blunt tip 1010 designed for entry into aperture 816 in blood vessel 810. In an exemplary embodiment, tip 1010 is sharp, (not shown) allowing it to pierce blood vessel 810 and/or modify aperture 816.

Additionally or alternatively, guide 1000 has one or more sharp edges along channel 1040 to aid in piercing blood vessel 810. Additionally or alternatively, the edges along channel 1040 are manufactured to be blunt to prevent modification of aperture edge 816 during insertion of channel 1040. In an exemplary embodiment, sharp tip is pressed into vessel 810 until wall 1020 contacts aperture edge 816, thereby properly modifying aperture edge 816 to form aperture 812 and/or positioning channel 1040 fully (and properly) in vessel 810.

In an exemplary embodiment of the invention, a sharp edge is defined between tip 1010 and wall 1020. Optionally, wall 1020 serves to define the length of the incision. Alternatively or additionally, the length of the cutting edge is selected to match a desired incision length.

In an exemplary embodiment, channel 2040 has a flare 1012 that defines, for example, a wider space than channel 1040 to facilitate movement of hooks 220 into channel 1040 without damage due to contact with channel 1040. Alternatively or additionally, flare 1012 prevents damage of hooks 220 through contact with surrounding tissue.

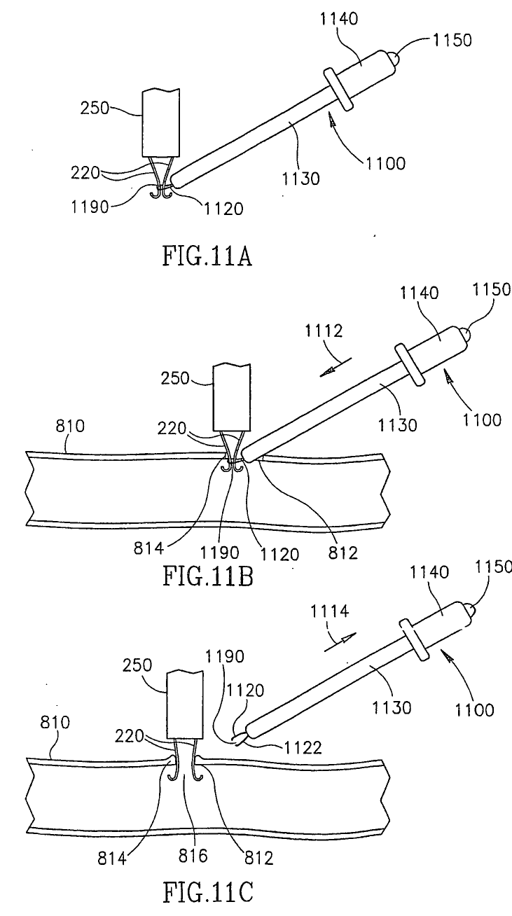

FIGS. 11A-C demonstrate the operation of a releasable grasper 1100 for grasping hooks 220 projecting from rotation sleeve 250 (or overtube 240), in accordance with an exemplary embodiment of the invention. In an exemplary embodiment, releasable grasper 1100 comprises a handle 1130 with one or more grasper wires 1120 and/or 1122 projecting from it. Grasper wires 1120 and/or 1122 comprise a grasper area 1190, adapted to grasp a plurality of hooks 220 during placement in blood vessel 810 without damaging hooks 220. Possibly, as hooks 220 are pressed together, they support each other, thereby providing strength against damage caused by inadvertent contact with vessel 810.

In an exemplary embodiment, grasper wires 1120 and/or 1122 are attached to grasper wire deployment button 1150. Upon pressing button 1150 on a grip 1140 in a direction 1112, grasper wires 1120 and/or 1122 move away from each other, expanding grasper area 1190 and thereby releasing hooks 220. Alternatively or additionally, the wires are retracted, possibly all the way into body 1130, thereby releasing hooks 220.

In an exemplary embodiment of the invention, one grasper wire, in the shape of a loop is used. Optionally, wires 1120 and/or 1122 remove from handle 1130, upon pulling grip 1140 in a direction 1114, without causing damage to hooks 220. In an exemplary embodiment, the loop tears when retracted into body 1130. A particular implementation is shown in FIG. 14 below.

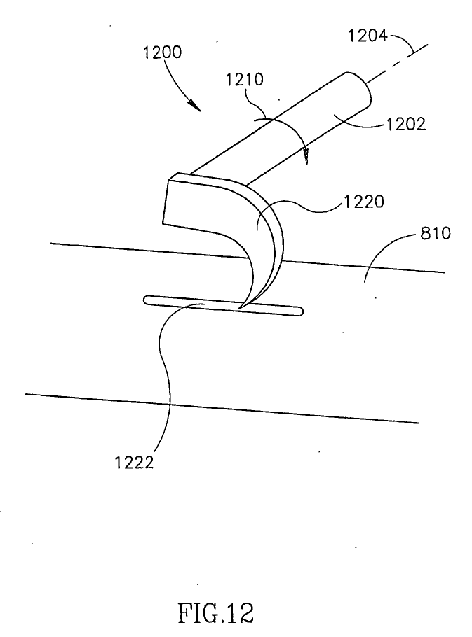

FIG. 12 is an isometric view of a blood vessel cutter 1200, shown cutting an aperture 1222 in blood vessel 810, in accordance with an exemplary embodiment of the invention.

In an exemplary embodiment, blood vessel cutter 1200 comprises a handle 1202 adapted to rotate in a direction 1210 around a longitudinal axis 1204.

Handle 1202 comprises a horn-shaped cutting edge 1220 perpendicularly connected to handle 1202. In an exemplary embodiment, upon rotating handle 1202 around longitudinal axis 1204, cutting edge 1220 rotates around handle 1202. When blood vessel cutter 1200 is held proximate to blood vessel 810, for example, by placing cutting edge 1222 along a longitudinal axis of blood vessel 810, cutting edge 1220 cuts an aperture 1222 into vessel 810 during rotation of handle 1202 in direction 1210. Aperture 1222, for example, is suitable for insertion of hooks 220 on anastomotic connector 101 (FIG. 1B).

In an exemplary embodiment, horn-shaped cutting edge 220 is of a length appropriate to cut aperture 1222 in a surface of blood vessel 810 that allows hooks 220 to enter vessel 810 easily and hook into the walls of vessel 810 around aperture 1222. Optionally, aperture 122 is cut into vessel 810 by vessel cutter 1200 during a single rotation in direction 1220 around axis 1204, said aperture 1222 comprising a longitudinal cut, the length of cutting edge 1222. Alternatively or additionally, cutter 1200 is rotated multiple times around axis 1210 and in relation to vessel 810. Optionally cutter 1200 is moved longitudinally along vessel 810 during said multiple rotations, thereby creating aperture 1222.

Depending on the particular procedural method followed, the target vessel may or may not be clamped or pressed against (e.g., using a finger) to prevent or reduce bleeding.

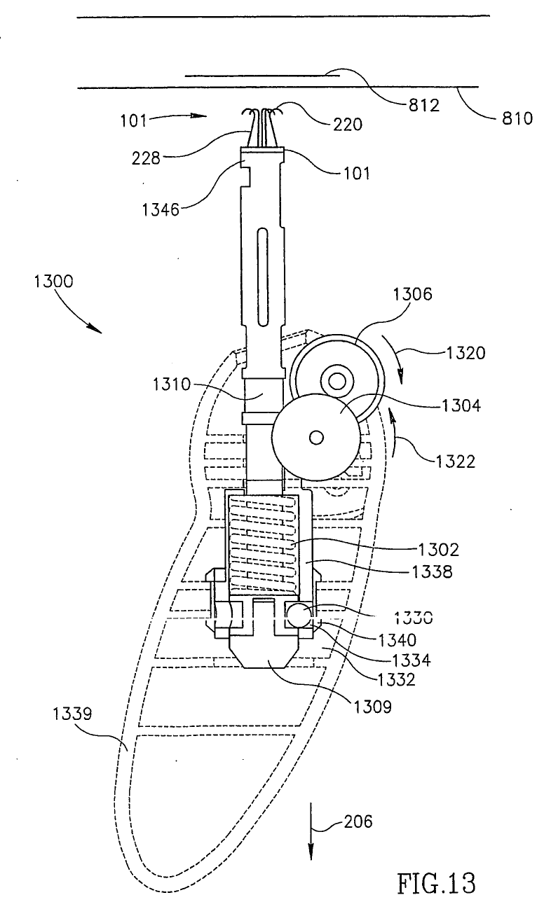

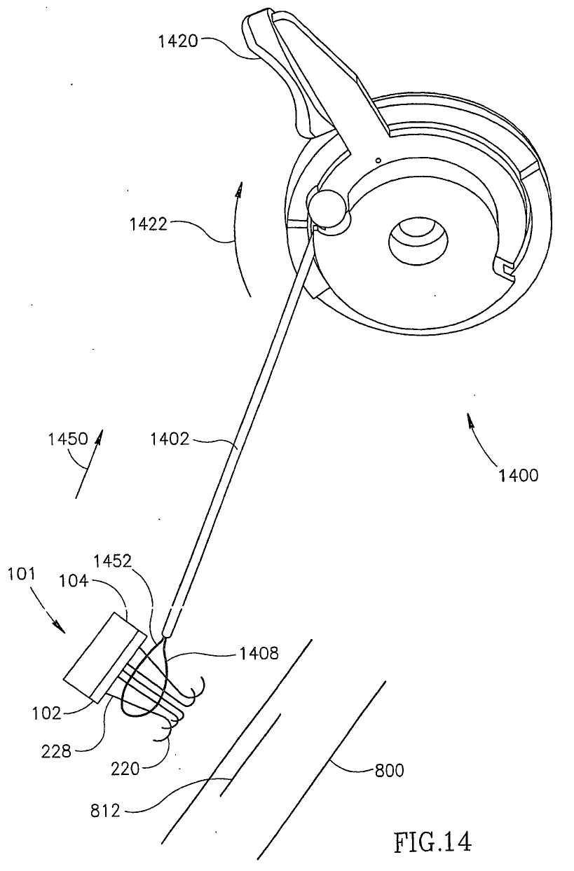

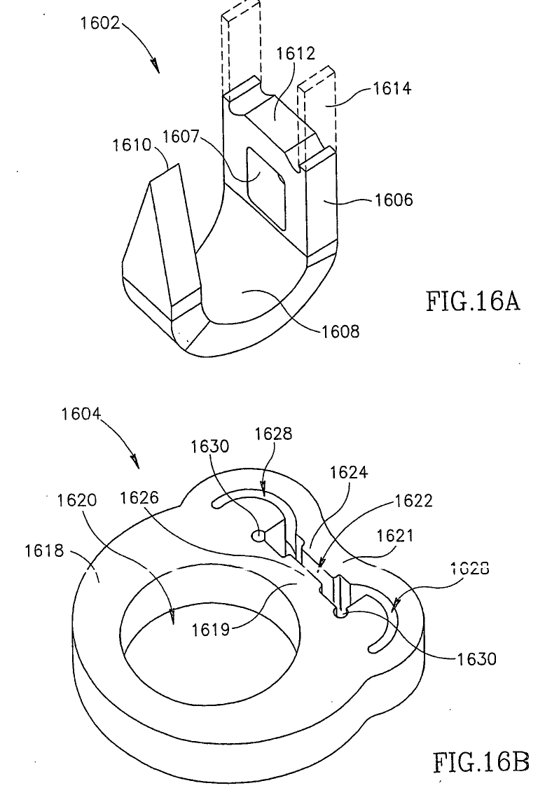

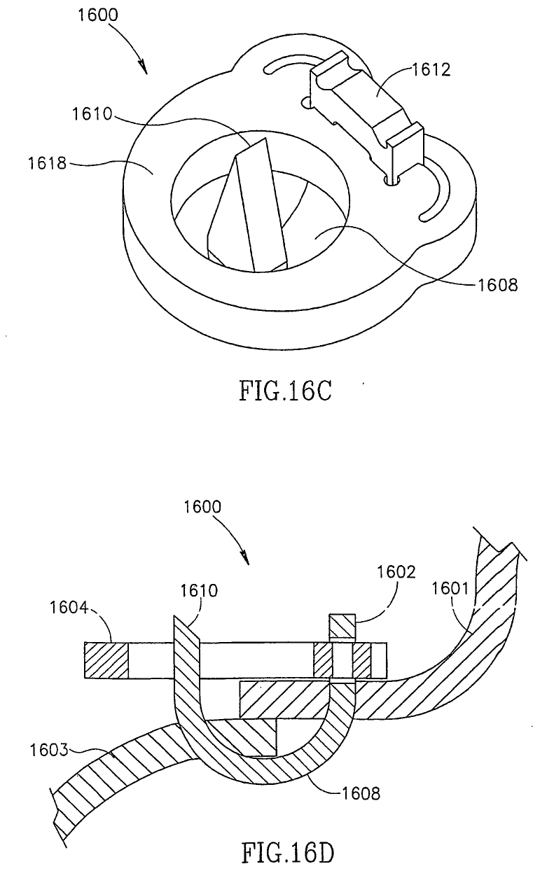

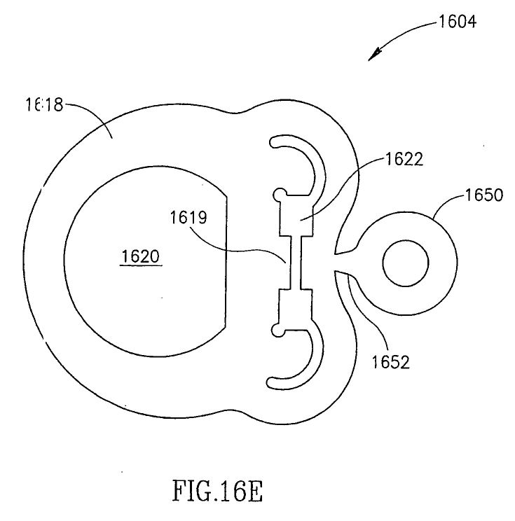

FIG. 13 is a partial cut-away view of a spring-loaded anastomosis connector delivery system 1300, which uses a pre-loaded spring for tearing extension sections (e.g., as described in FIG. 16 below) off of hooks 220 and advancing the deployment of an anastomotic connector.

In an exemplary embodiment of the invention, a manual retraction device, for example a knob 1306 (and an optional associated gear 1304) are used to retract a tube 1310 which pulls back on hooks 220. The hooks are prevented from moving by ring 102 being held in place, for example, by a non-moving tip 1346 of system 1300. This pulling back also releases an interlock mechanism, thereby releasing spring 1302. In an exemplary embodiment of the invention, a base 1309, optionally with a shock absorbing function, interconnects tube 1310 and spring 1302. However, as long as the interlock is in place, spring 1302 is constrained by a housing 1338. In an exemplary embodiment of the invention, knob 1306 is actually used to push retract housing 1338 and with it base 1309 and tube 1310. Other coupling methods may be used as well.

In an exemplary embodiment of the invention, the interlock mechanism comprises a ball 1330 which is constrained by a sleeve 1340. However, ball 1330 moves back with housing 1338, until sleeve 1340 no longer constrains it and it is released into a space 1332 formed in a body 1339 of system 1300. This releases the constraining of spring 1302, applying an impulse force to base 1309 and tearing the extensions off of hooks. In an exemplary embodiment of the invention, system 1300 is designed so that the interlock is released only after the hooks are locked in place (e.g., as in FIG. 16C).

Alternatively or additionally, one or more levers 230 and/or 232 (shown in FIG. 2A) govern the movement of tube 1310. Spring 1302 may be of various types, for example, comprises a coiled wire, a ribbon of tempered material, for example spring steel and/or a pneumatic force-providing pump.

FIG. 14 is a cut-away view of breakable spike grasper device 1400, adapted to grasp multiple connector spikes 228 on a connector 101 in relation to a blood vessel aperture 810 and to breakaway from said spikes 228 upon pulling grasper device 1400 away from spikes 228. In an exemplary embodiment, grasper devise 1400 comprises at least one spike grasper wire 1408 that encircles spikes 228, so that spikes are properly grasped as connector 101 is placed in an aperture 1222 in vessel 810, thereby allowing hooks 220 to be positioned for deployment in blood vessel 810.

In an exemplary embodiment, spikes 228 are maintained by grasper device 1400 in a cone configuration so that hooks 220 rest against each other. A cone configuration of spikes 220, for example, provides greater stability of spikes 228 than when spikes 228 are not resting against each other. Additionally or alternatively, spikes 228 are manufactured to be more rigid so that they are less prone to being damaged by inadvertent brushing against biologic tissue. In an exemplary embodiment, spike grasper wire 1408 of breakable grasper device 1300 extends from a handle 1402 and is retracted in a direction 1450 by movement of a shifter 1420. As shifter 1420 is moved in a direction 1422, grasper wire 1408 is pulled in direction 1450, compressing spikes 228 against each other.

Grasper wire 1408, for example, comprises at least one breakaway area 1452 that breaks when grasper wire 1408 is pulled away in direction 1450 away from compressed spikes 228. When grasper wire 1408 break away from spikes 228, spikes 228 are released, for example, in aperture 1222 of vessel 810, so that hooks 220 can be deployed therein.

Optionally, breakable spike grasper device 1400 is used in conjunction with an anastomotic connector 101 constructed with spikes 228 that withstand the breakaway force used to break grasper wire 1408 from connector 101.

In an exemplary embodiment of the invention, shifter 1420 has three resting positions, a first one where the loop 1408 is open for easy insertion of the hooks, a second one where the loop is made smaller and the hooks are compressed and a third one where the loop is compressed even more and is thus torn and the hooks released.

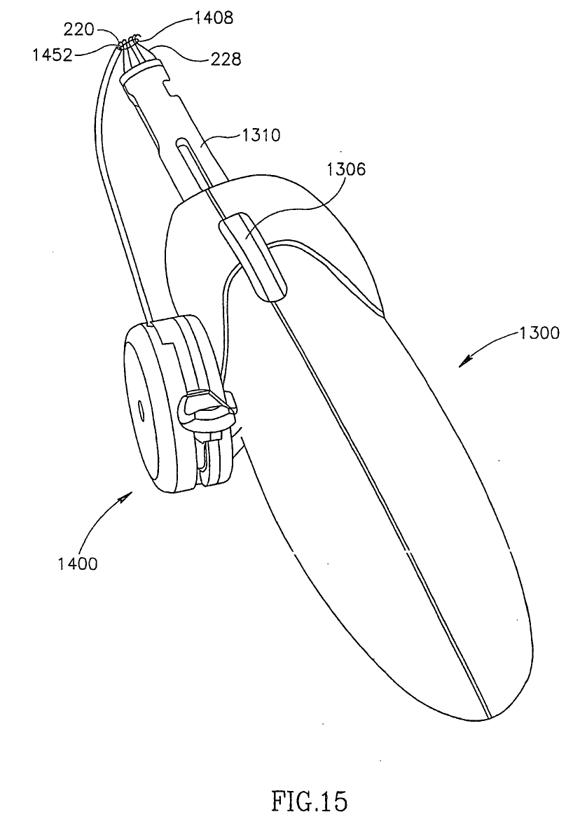

FIG. 15 is an isometric view of the delivery system of FIG. 13 with the spike receptacle of FIG. 14 mounted on it. Various of the assistance devices, such as the hook arranger and the guide may be, for example permanently mounted or temporarily mounted on a delivery system, alternatively or additionally, to the receptacle of FIG. 14.

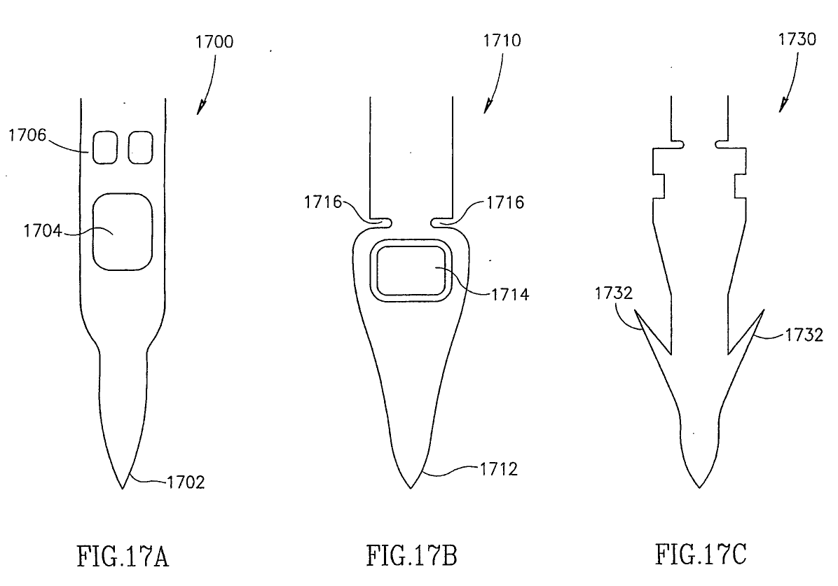

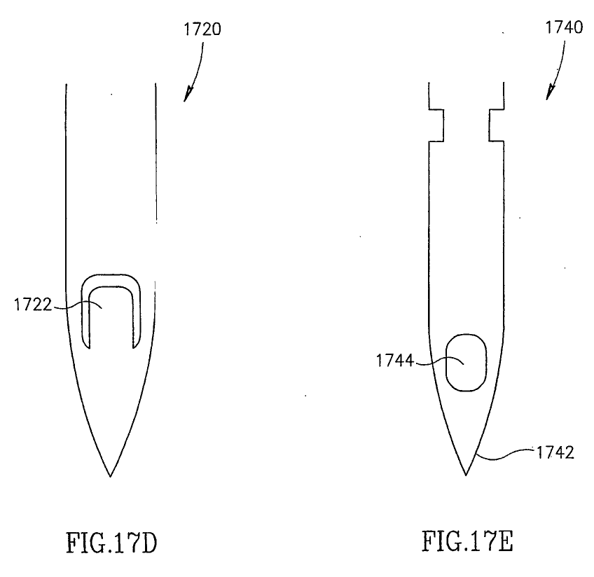

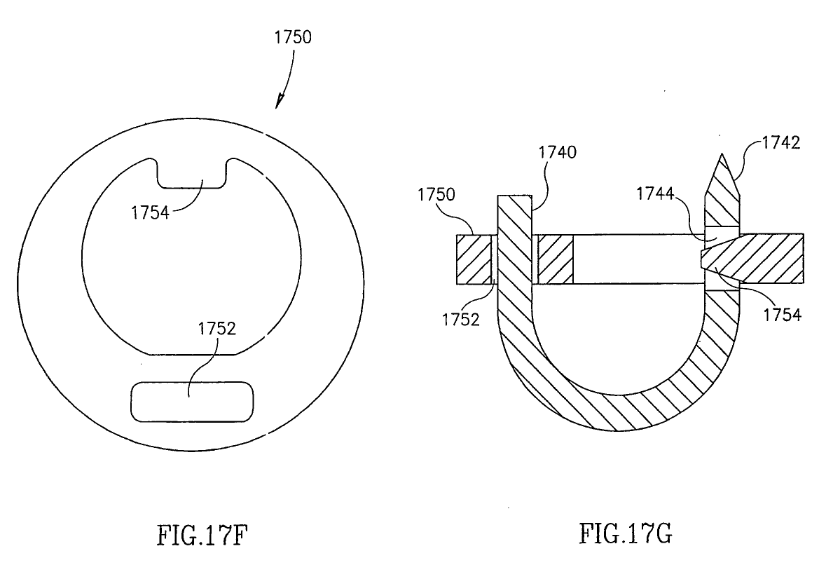

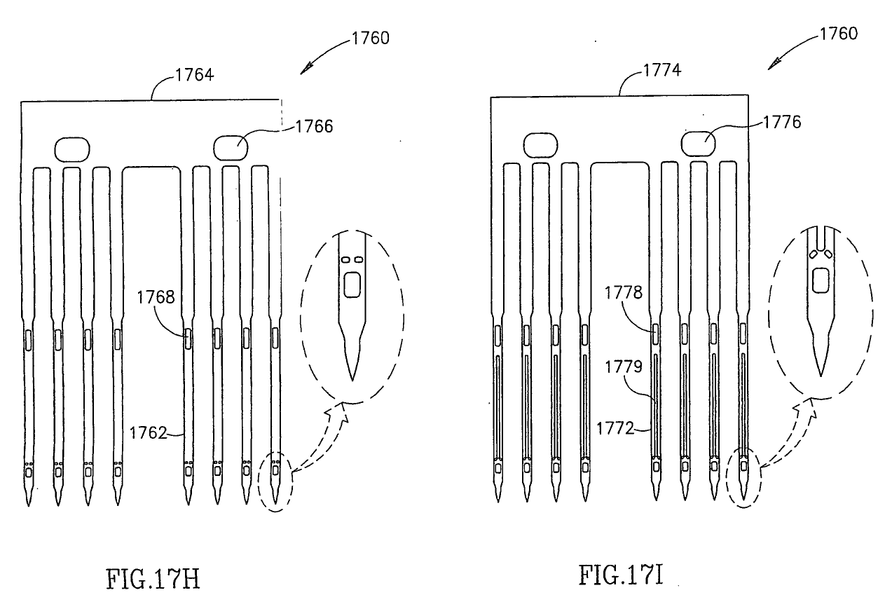

FIGS. 16A-16E illustrate an interlocking anastomotic connector portion 1600, in accordance with an exemplary embodiment of the invention. This portion may be part of a complete anastomotic connection, for example using multiple such portions. FIG. 16A shows a hook segment 1602 (FIG. 17I shows a set of such segments), FIG. 16B shows an eye segment 1604 (of which a plurality are optionally provided) that interlocks with hook segment 1602 and FIG. 16C shows an interlocked connector portion 1600. FIG. 16D is a side cross-sectional view of a deployed portion 1600, showing how a graft 1601 and a coronary artery 1603 are interconnected by connector portion 1600.

Referring to FIG. 16A, hook 1602 comprises an interlocking section 1606 and a tissue holding portion 1608. In the embodiment shown, interlocking section 1606 comprises an aperture 1607 into which two tabs enter to look hook segment 1602 to eye segment 1604. In other embodiments, only a single tab is used. In this embodiment tissue holding section 1608 includes a tissue penetration tip 1610, which transfixes graft 1601 and later, during deployment, coronary vessel 1603 (or any other target vessel).

In an exemplary embodiment of the invention, hook segment 1602 is originally connected to an extension and is torn off during deployment, for example at a tearing plane 1612, described below. Dotted lines 1614 indicate this extension.

Referring to FIG. 16B, in an exemplary embodiment of the invention, eye segment 1604 comprises a general ring shaped body 1618 defining an internal aperture 1620. Optionally, tip 1610 passes through aperture 1620, however, this is not essential. Further, if tip 1610 is not sharp, is short and/or is limited in its penetration (e.g., being fork shaped), it will not reach the top and/or bottom plane of eye segment 1604. Positional alignment of the axis of tip 1610 and aperture 1620 is also not essential. Optionally however, such alignment is generally provided, to prevent the connector portion from slipping off the blood vessels. Optionally, tip 1610 transfixes or at least penetrates both blood vessels.

While a closed ring shape is shown, this is not essential but may be useful to prevent undesirable tearing of the blood vessels by projections of eye 1604.

In the embodiment shown, two tabs 1624 and 1626 are defined to engage aperture 1607, from either side, when hook 1602 passes through an aperture 1622 of eye segment 1604 and aperture 1607 is suitably aligned. In an exemplary embodiment of the invention, the two tabs are mounted on spring elements, for example, a section 1621 of body 1618 and a separate spring element 1619. As shown, a pair of channels 1628 are used to separate the two spring elements, to allow distribution of elastic distortion over relatively large areas, and thus allow it to act as a spring. In an exemplary embodiment of the invention, the spring elements are pre-stressed to be in a closed position. A pair of strain relief holes 1630 are also shown. Holes 1630 are also useful for some types of machining methods to ensure that the comer is at least complete and prevent binding of the hook element in the channel. Other exemplary spring element designs are shown below. In an exemplary embodiment of the invention, the distance between tabs 1624 and 1626 is designed to be less than a thickness of hook segment 1602.

FIG. 16E shows a variation of eye segment 1604. In some cases it may be difficult to insert hook segment through its channel 1622. In an exemplary embodiment of the invention, channel 1622 is widened by separating spring elements 1619 and 1621. In an exemplary embodiment of the invention, a ring or other engagable element 1650 is attached to eye segment 1604. During insertion of hook segment, this ring is pulled back, for example it being held using tweezers or being threaded with a string that is pulled back. After loading, ring 1650 may be cut off or tom off, for example at a pre-defined weakened point 1652 FIGS. 17A-17I illustrate several variations of hook segment 1602, in accordance with exemplary embodiments of the invention.

FIG. 17A shows a flattened view of a hook segment 1700, including a sharp tip 1702, a locking aperture 1704 and a pair of weakening apertures 1706 at which segment 1700 tears when deployed. Alternatively a single weakening aperture may be provided. It should be noted that the apertures may be defined at an angle to the axis of hook segment 1700, for example, to guide tearing strain or to enable the weak areas to be made longer in a limited space. One potential advantage of using a single tearing aperture is that a larger tearing stop may be inserted into a single aperture. One potential advantage of using a pair (or more) of apertures is that they can be aligned off of the locking apertures and/or made small enough so that they do not inadvertently engage the locking tabs of the eye segment. FIG. 17B shows an example where a pair of slots 1716 are provided in a hook segment 1710. Optionally, this allows the locking tabs of the eye segment to come from a direction perpendicular to the hook plane (e.g., if it is made of sheet metal). Thus, one or both of the locking aperture and the tearing aperture may be implemented as one or more slots, in some embodiments of the invention.

It should be appreciated that in some embodiments of the invention, the interlocking is used not only to prevent the eye and hook segments from separating but also as a stop that holds the hook segments against the eye segment while an extension of the hook element is pulled and torn off. In other embodiments of the invention, a separate holding means is provided.

FIGS. 17C and 17D show various on the tip of the hook. In FIG. 17D, a tab 1722 is defined in a hook 1720, which may assist in preventing tissue from slipping off. In FIG. 17C, one or more side barbs 1732 are defined in a hook 1730.

FIGS. 17E shows a hook segment 1740 having a locking aperture 1744 near its tip 1742. FIG. 17F is a top view of a suitable eye segment 1750 for hook segment 1740, showing a aperture 1752 for passage of hook 1740 through the eye and a locking tab 1754 for engaging aperture 1742. FIG. 17G is a side cross-sectional view of a deployed connector portion. Optionally, a locking mechanism (e.g., using an interlocking tab aperture pair) at the base of hook segment 1740 is also provided (not shown).

Optionally, the mechanism of FIG. 17D is used for locking the eye to the hook at one or both ends of the hook.

FIG. 17H shows a complete set of hooks 1760, in plan view for use in anastomotic connections, in accordance with an exemplary embodiment of the invention. A ring element 1764, on which a plurality of hook segments 1762 are mounted, includes a plurality of apertures 1766 used for pulling back the hook for tearing. An aperture 1768 is optionally provided, for example for holding and/or as a heat darn or heat sink attachment during heat treatment of the hook segments. In an exemplary embodiment of the invention, set of hooks 1760 is mounted on a plurality of eye segments before the tips of the hooks are bent. After bending, (e.g., during the manufacturing process) the tips are heated. This heating might cause a weakening of the spring action of the eye segments, which is why the apertures are provided. Alternatively or additionally, the apertures are provided as a resting location for the tabs, so that the eye segments are in a configuration that can benefit from the heat treatment, or at least not be damaged.

FIG. 17I shows a complete set of hooks 1770, in plan view for use in anastomotic connections, in accordance with another exemplary embodiment of the invention. A ring element 1774, on which a plurality of hook segments 1772 are mounted, includes a plurality of apertures 1776 used for pulling back the hook for tearing. An aperture 1778 is optionally provided, for heat treatment, as described above. An elongated slot 1779 is optionally provided for engaging an extension guiding and tearing mechanism (mushroom) described below.

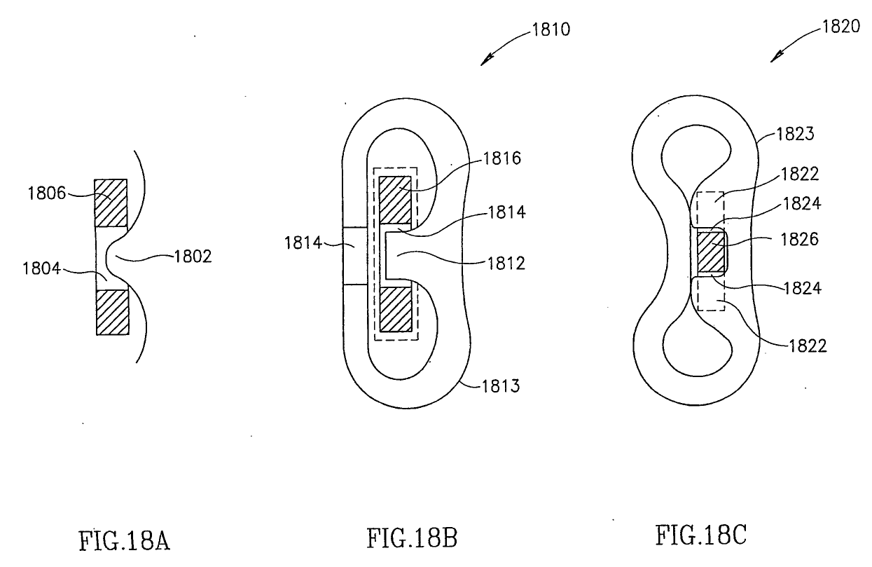

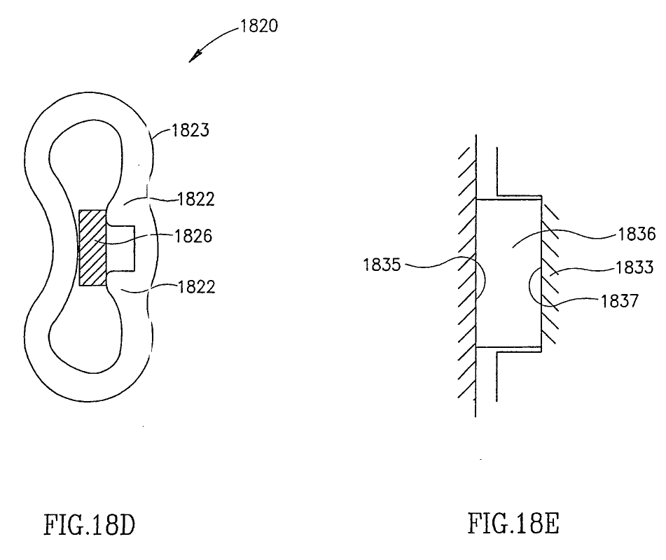

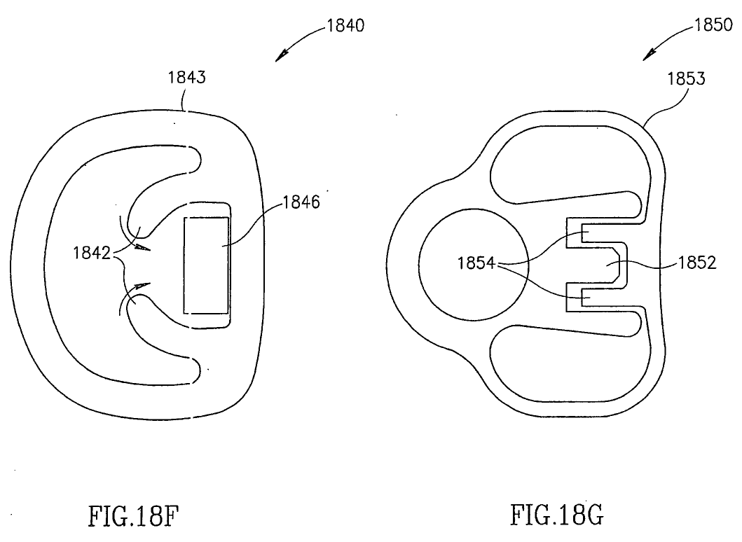

FIGS. 18A-18G illustrate various locking mechanisms, all of which are optionally in the plane of the eye segment, in accordance with an exemplary embodiment of the invention. All these FIGS. are shown in top cross-sectional views at the plane of the eye segment.

FIG. 18A shows a bump-tab 1802 which enters a small distance into an aperture 1804 of a hook section 1806. Optionally, instead of an aperture, a depression may be provided.

FIG. 18B shows a tab interlocking mechanism in which a tab 1812 of an eye segment 1813 passes through an aperture 1814 of a hook segment 1816. It should be noted that the entire segment 1813 compresses in this example, and does not maintain a ring shape. During this distortion the tip of tab 1812 may contact or slide above or below a reference 1814. Optionally, the ring structure at this point is removed or made into a receptacle for the tip of tab 1812. FIG. 18C shows a slot based locking mechanism in which two slots 1824 are cut in the sides of a hook segment 1826, and which receive tab portions 1822 of an eye segment 1823. Alternatively to specially defined tabs, the body of eye segment 1823 may be slotted to receive hook segment 1826. FIG. 18D shows the situation when slots 1824 are not aligned with tab portions 1822. FIG. 18E shows a friction based locking, where a portion of a hook segment 1836 is optionally roughed so that it is fractionally engaged by two faces 1835 and 1837 of an eye segment 1833.

FIG. 18F shows an eye segment 1843, in which two tabs 1842 press in a direction of the shown arrows against a hook segment 1846. The engagement may be, for example by friction or by interlocking. For clarity, the tabs are shown not touching. Also in other FIGS. spaces are shown for clarity where in reality there are no spaces, for example between blood vessels and hooks.

FIG. 18G shows an eye segment 1850 including opposing locking tabs 1852 (only one shown in this embodiment) from one direction and 1854 which extend from an opposite direction. Alternatively, one or more of the tabs may be used to push the hook segment (not shown) against the opposing tab. Possibly, by using tabs 1854 as springs to push the hook segment aperture onto tab 1852, tab 1852 can be made more with a better match to its respective aperture. For example, stiffening tab 1852 so no (or reduced) out of plane motion is possible, allows its edges to be rectangular, rather than inclined, as might be desirable in other tabs to assist sliding of the tab into its respective aperture.

It should be noted that while the locking methods of FIGS. 18A-18G are all interlocking, in that the locking is by a single mechanism and independent of the existence of tissue, some embodiments of the invention, for example as shown in FIG. 17D are not interlocking, nor are they in the plane of the eye segment.

In an exemplary embodiment of the invention, the interlocking mechanism used is spring based, in that a tab is elastically (including shape memory and super-elastically) disposed to go in a certain direction, which is only available when the hook segment is properly aligned with the eye segment. Alternatively, a plastically deforming mechanism, for example with external clamp jaws that compress the eye segments, may be provided. optionally, a layer is provided between the eye segments and the side vessel, so that they are sandwiched between two layers and their distortion is prevented. Such a sandwich layer is described, for example in WO 02/30172, the disclosure of which is incorporated herein by reference.

The spring mechanism can be of various types. In particular, a long spring is generally useful for providing some flexibility in positioning and/or for distributing stress. A shorter spring, on the other hand, may be useful in preventing undesired motion along the axis of the hook segment. Various tradeoffs may be selected, for example based on the particular blood vessels being attached to each other.

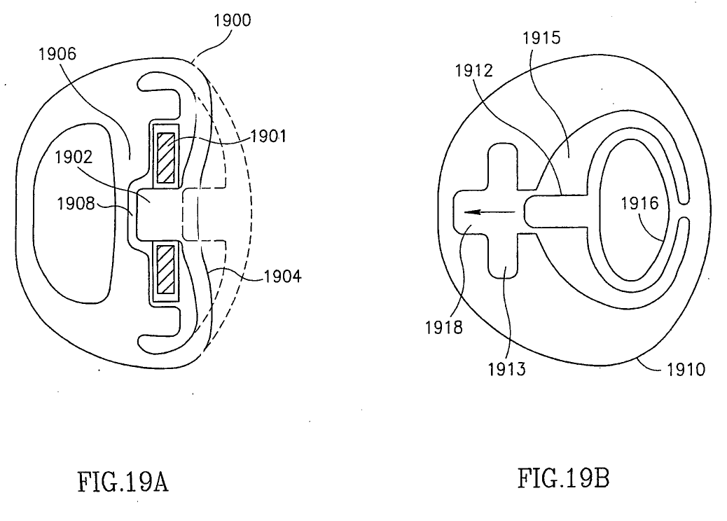

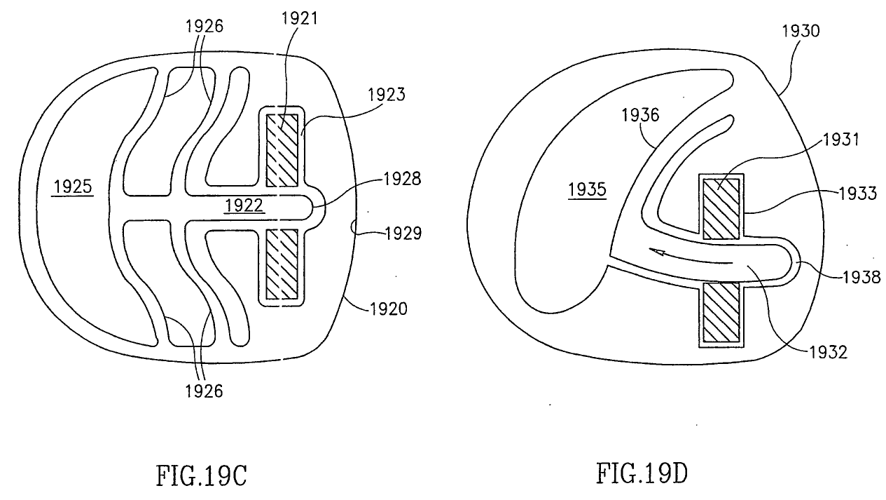

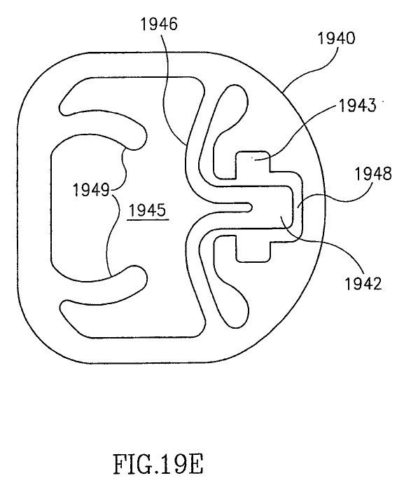

FIG. 19A shows an eye segment 1900, in which a tab 1902 is mounted on an outer portion 1904 of the segment. An internal strut 1906, optionally with a receptacle 1908 for tab 1902 is optionally provided to prevent escape of a hook segment 1901.

FIG. 19B shows an eye segment 1910, in which a spring 1916 is mounted on a far side of eye segment 1910 opposite from a receptacle 1918 for a tab 1912. A slot 1913, for a hook segment (not shown) is provided in the body of eye segment 1910. The shape of an opening 1915 formed within eye segment 1910 and within which spring 1916 is mounted may vary, for example, be circular, oval or triangular. In an exemplary embodiment of the invention, the shape is selected to ensure minimum rigidity of the eye segment, while minimizing the amount of foreign material in the body. It should be noted that in some embodiments of the invention, the eye segment itself collapses (e.g., a small part thereof, such as the part near slot 1913, a large part thereof, such as the part near opening 1915, or the whole segment, for example as shown in FIG. 18).

FIG. 19C shows an eye segment 1920, in which a tab 1922 is mounted on two sets of springs 1926 that are wholly internal to eye segment 1920, so that segment 1920 does not distort during deployment. Alternatively, only one set of springs is used, however, the use of two sets may improve the planar stability of tab 1922. A receptacle 1928 is optionally provided in the body of eye 1920. Optionally, receptacle 1928 is continued so that it transects the body of eye segment 1920, at a point 1929.

FIG. 19D shows an eye segment 1930, in which a single (internal) spring 1936 is used to advance a tab 1932. This segment is asymmetrical, which may be an advantage in allowing the spring mechanism and/or metal portions of the eye segment to be distanced from the anastomosis location. Alternatively or additionally, by providing a single leaf spring a more elastic spring may be possible.

For this an other springs, the spring element is optionally selectively treated, for example, by heat or chemical treatment, or thinned, to make it more or less elastic, as desired.

Alternatively or additionally, the rest of the eye segment may be treated to make it more rigid.

FIG. 19E shows an eye segment 1940, in which a short pair of springs 1946 are used for a tab 1942. Optionally, one or more tabs 1949 are provided in an opening 1945 defined by eye segment 1940, for example to prevent ingress of tissue and/or improve planar stability of the eye segment during and/or after anastomosis.

It should be noted that the description and FIGS. show generally perpendicular slots and/or apertures in the hooks segment and/or eye segments. However, this is not essential. For example, a slot 1943 for a hook segment in eye segment 1940 may be oblique to the plane of the eye segment. Alternatively or additionally, the aperture (of the hook segment) into which tab 1942 fits can be angled, for example anticipating forces applied in a certain direction and/or to assist in entry of the tab into the aperture.

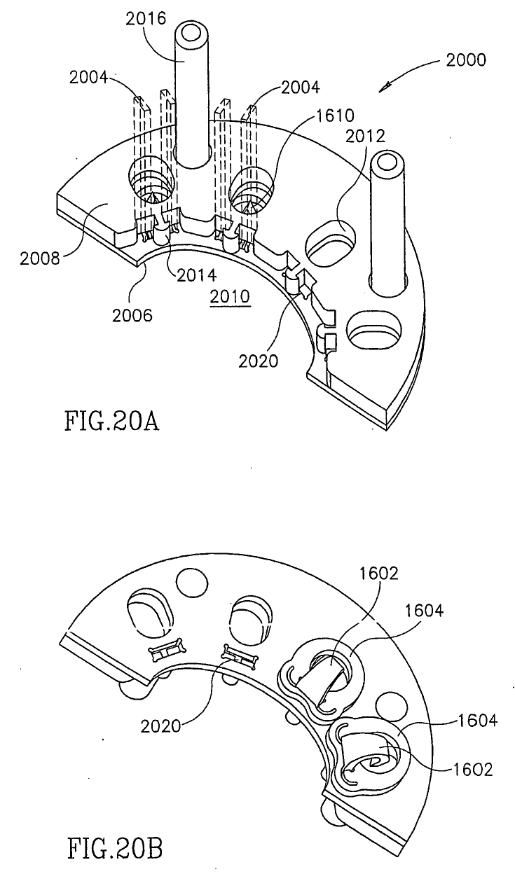

FIGS. 20A-20B show a deployment mechanism 2000 for the connector portion of FIGS. 16A-16D, in accordance with an exemplary embodiment of the invention. A complete connector comprises a plurality of eye segments 1604 and a plurality of hook segments 1602, which may be interconnected as shown, for example in FIG. 17I. In some embodiments of the invention, the eye segments are also interconnected, for example as shown in WO 01/70090 and WO 02/30172, the disclosures of which are incorporated herein by reference. If the eye segments are not interconnected, a same connector is optionally usable for a wide range of vessel sizes, possibly depending on the spike delivery system to align the hook tips correctly with the incision.

In an exemplary embodiment of the invention, mechanism 2000 is placed at the end (e.g., on the tip of overtube 240 of capsule 400) of the delivery system, for example as described in FIGS. 2-15. With respect to those FIGS. it should be noted that system 200, with suitable modifications may be used for a range of connectors, including single part connectors and connectors that are two parts or more parts, like those of FIGS. 1 and 16. For deployment, an extension 2004 of each of the hook segments is retracted away from the blood vessel, so that it tears, at plane 1612 (FIG. 16). In an exemplary embodiment of the invention, two layers are provided, a forward layer base 2006, on which the eye segments rest, and are optionally attached, for example using a weak glue, or sucrose, and a back layer 210, used to tear the hooks segments. Only half of a delivery mechanism is shown, with an aperture 2010 being used for passage of a graft through the delivery mechanism. In an exemplary embodiment of the invention, a plurality of poles 2016 are provided for attaching the base layer to the delivery system. A plurality of apertures 2021 are optionally provided to receive tissue and/or tips 1610.

In an exemplary embodiment of the invention, a plurality of mushroom shaped protrusions 2014 are mounted on extensions 2004, and incidentally may be used to stabilize the hook segments, against bending. Other shape protrusions, such as even width fingers or spikes may be used instead. The mushroom shape may also b useful in preventing damage to the graft. When extensions 2004 are pulled back, the bottom of each mushroom protrusion contacts plane 1612 and then the hook extension tear at that point.

FIG. 20B is a bottom view, showing a plurality of mounted connectors (after tearing, but with no graft shown, for improved visualization) and also affording a better view of a plurality of apertures 202 through which hook segment extensions 2004 are provided. While the above description has provided one eye segment for one hook segment, this is not essential, for example an eye segment may include channels and/or locking mechanisms for two or more hook segments. Alternatively or additionally, even after deployment, two or more hook segments may remain connected.

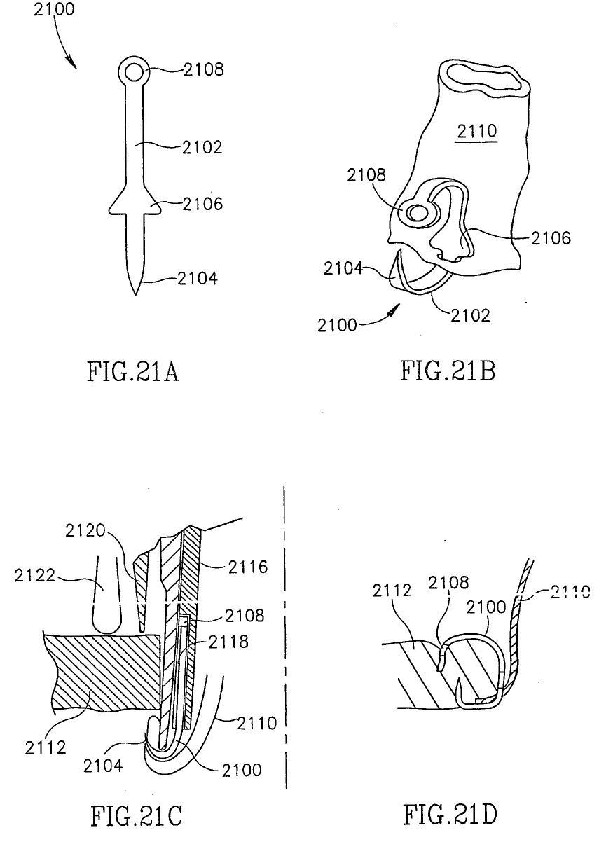

FIGS. 21A-21D illustrate a spike clip 2100 in accordance with an exemplary embodiment of the invention. spike clip 2100 includes a body 2102, a tissue penetration tip 2104, a tissue penetration stop 2106 and a back tip 2108 which may or may not be sharp. A blunt embodiment is shown.

FIG. 21B shows spike clip 2100 deployed on a graft 2110, with no side vessel shown. In an exemplary embodiment of the invention, the spike is super-elastic, elastic or shape memory and is pre-stressed to this geometry.

FIG. 21C shows an exemplary apparatus for deploying a plurality of spike clips (only one shown), to perform an end-to-side anastomosis. Graft 2110 is everted over spike clip 2100 and penetrated by its tip 2104. Spike clip 2100 is prevented from bending because it is held between an inner contra tube 2116 and a delivery tube 2114. A recess 2118 is optionally defined in contra tube 2116, optionally matching the profile of spike clip 2100, for example recess 2118 including a bump to engage stop 2106 and/or back tip 2108. In operation, the graft vessel is inserted into a target side vessel 2112 and retracted until tip 2104 engages the side vessel. An outer tube 2120 is optionally provided to prevent motion of side vessel 2112. Then, delivery tube 2114 is retracted, releasing back tip 2108. An optional holding tube 2112 is also shown.

FIG. 21D shows a deployed spike clip, in side cross-sectional view. In other embodiments, back tip 2108 is more nearly parallel to side vessel 2112. It should be appreciated that spiked clips may also be used for side-to-side connections. For example, the clip of FIG. 4 of WO 02/30172, may be used for side to side connections.

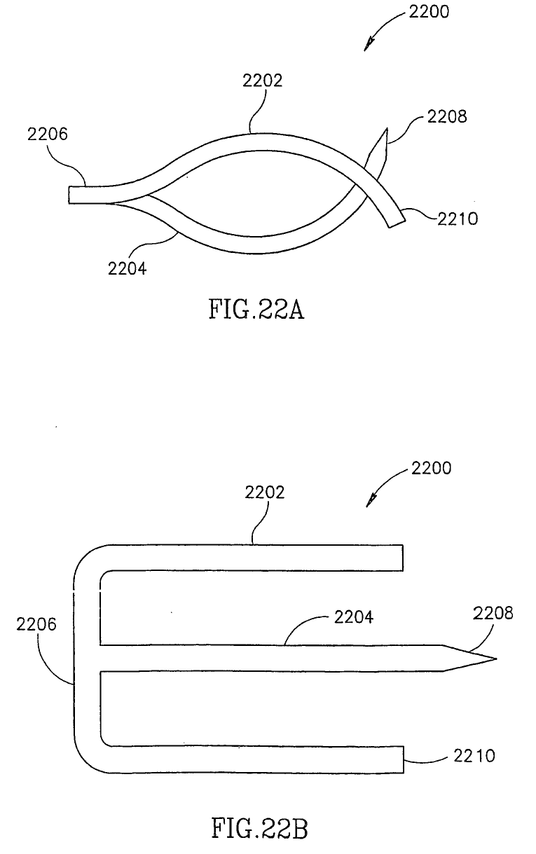

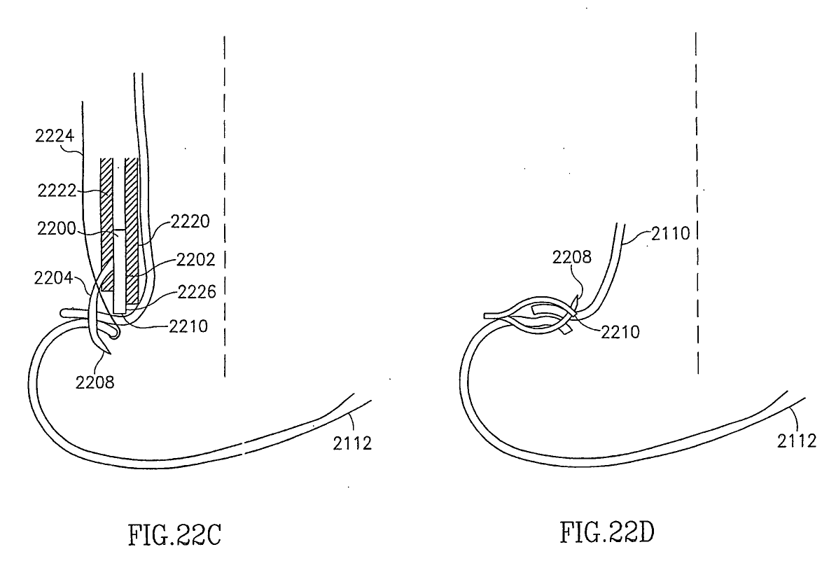

FIGS. 22A-22D illustrate a spiked clip 2200 which stitches two blood vessel lips together by transfixing a first vessel, transfixing a second vessel and then optionally transfixing the first. Another potential advantage of this clip is that the lips of the side vessel (e.g., a diseased and weakened coronary vessel) are minimally distorted in some implementations.

FIG. 22A is a side view and FIG. 22B is a top view of clip 2200, which is optionally elastic, super elastic or shape-memory, FIG. 22B is a top view. In the design shown, a base bar 2206 has mounted on it one bottom spike 2204 with a sharp tip 2208 and two straddling top spikes 2202 with blunt tips 2210. Alternatively, sharp and/or forked tips are provided for tips 2210. Base 2206 may have other shapes, for example, arcuate. In an embodiment where plastic deformation of clip 2200 is used for deployment, one or more tabs (not shown) are optionally provided on clip 2200, so that by relative motion of the tabs and/or the device, the relative position of the spikes can be changed.

FIG. 22C shows clip 2200 during deployment with an exemplary deployment mechanism. A plurality of clips 2200 (one shown) are held between an inner tube 2220 and an outer tube 2222. As in other embodiments, the term tube is used to describe the general arrangement. An actual tube is not strictly essential, only convenient, and could be replaced, for example, by a series of elongate clip holders. Graft 2110 is everted over the clips so that spike 2204 transfixes the graft. The lips of side vessel 2112 are also transfixed on spike 2204, for example using a puller mechanism 2224, such as described in WO 01/41624, WO 01/70090 and WO 02/30172, the disclosures of which are incorporated herein by reference. Spike 2204 is optionally provided through a channel in tube 2222. In an exemplary embodiment of the invention, spike 2202 is held straight by a receptacle area defined between tubes 2220 and 2222. This provides a spacing between the spikes. Optionally tube 2220 includes a lip 2226 in which tip 2210 rests.

In FIG. 22D, clip 2200 is rotated so that it lies flat on side vessel 2112 and tip 2208 can optionally penetrate graft 2110 again. The release of clip 2200 may be effected, for example, by retracting tube 2222. Clip 2200 will then rotate around lip 2226 and reach the position as shown.

The PCT applications mentioned in the related application section as of the filing in the PCT of this application contain various delivery system and connector elements which may be practiced in conjunction with the embodiments described in the present application.

The above devices may be varied in various ways for adaptation for specific types of blood conduits. For example, larger devices may be designed for use in large blood vessels and/or softer coatings, for example a silicone rubber coating, may be applied to devices designed for used in conduits with delicate walls. In some embodiments of the invention, a device is packaged and/or sold with an instruction leaflet, describing the device dimensions and/or situations for which the device should be applied. The devices may be used, for example for aortic connections, for coronary vessels and with various types of grafts, including, for example, artificial grafts, xenografts, harvested veins, harvested arteries and in-situ arteries, such as the LIMA and RIMA. The dimensions, elasticity and/or strength of the various elements, for example hooks and eyes may be adapted for various situations, or, for example, as noted, one size may be sufficient for a wide range of situations. For example, the design may take into account blood vessel strength thickness, compressibility, incision size, and diameter.

It will be appreciated that the above described methods and devices of vascular manipulation may be varied in many ways, including, changing the order of steps, the order of making the anastomosis connection., the order in each anastomosis, the exact materials used and/or design of the anastomotic connectors and/or installation devices.

Further, in the mechanical embodiments, the location of various elements may be switched, without leaving the aspect of the invention, for example, switching moving elements for non-moving elements where relative motion is required. In addition, a multiplicity of features, and devices have been described. It should be appreciated that different features may be combined in different ways. In particular, not all the features shown above in a particular embodiment are necessary in every similar exemplary embodiment of the invention.

Further, combinations of the above features, from different described embodiments are also considered to be within the scope of some embodiments of the invention. In addition, some of the features of the invention described herein may be adapted for use with prior art devices, in accordance with other embodiments of the invention. The particular geometric forms used to illustrate the invention should not be considered limiting the invention in its broadest aspect to only those forms, for example, where a circular lumen is shown, in other embodiments an oval lumen may be used.

Also within the scope of the invention are surgical kits comprising sets of medical devices suitable for making a single or a small number of anastomosis connections. Measurements are provided to serve only as exemplary measurements for particular cases. The exact measurements applied will vary depending on the application. When used in the following claim, the terms “comprises”, “comprising”, “includes”, “including” or the like means “including but not limited to”.

It will be appreciated by a person skilled in the art that the present invention is not limited by what has thus far been described. Rather, the scope of the present invention is limited only by the following claims.

1. A transfixing assistance device for transfixing a blood vessel on one or more hooks of an anastomotic connector, said transfixing device comprising:
a handle defining a longitudinal axis; and
an extension projecting from said handle comprising an orifice at its end, said extension being offset at an angle to said longitudinal axis so that, when said handle is rotated said end circumducts at a radius suitable for everting a graft over a spiked anastomotic connector.

2. A transfixing assistance device according to claim 1, wherein said orifice is adapted to transfix blood vessel tissue on said one or more connection hooks without damaging said tissue.

3. A transfixing assistance device according to claim 1, wherein said device comprises two or more opposable extension projection from said handle, arranged to function as a forceps.

4. A transfixing assistance device according to claim 1, wherein said orifice comprises a closed aperture.

5. A transfixing assistance device for transfixing a blood vessel on one or more hooks of an anastomotic connector, said transfixing device comprising:
two elongate members attached at a base thereof; and
an orifice adapted to transfix blood vessel tissue on said one or more connection hooks without damaging said tissue, defined on at least one of said members.

6. A method of guiding hooks of an anastomotic connector into an aperture of a blood vessel, comprising:
surrounding said hooks with a mechanical element that compresses them towards each other;
inserting said compressed hooks into an aperture of a blood vessel; and
releasing said hooks.

7. A method according to claim 6, wherein surrounding comprises protecting said hooks from tissue adjacent said vessel.

8. A method according to claim 6, wherein surrounding comprises inserting said hooks into a guide.

9. A method according to claim 6, wherein surrounding comprises inserting said hooks into a hook arranger.

10. A method according to claim 6, wherein surrounding comprises inserting said hooks into a wire loop.

11. A hook arranging device for arranging a plurality of anastomotic hooks projecting from an anastomotic connector, said hook arranging device comprising:
one or more arranging plates adapted to arrange a plurality of hooks of an anastomotic connector; and
a coupler adapted for coupling said at least one plate to an anastomotic connector delivery system.

12. An arranging device according to claim 11, wherein said one or more plates are removably connected to said delivery system.

13. An arranging device according to claim 11, wherein said one or more plates comprise two edges that presses said plurality of hooks between them.

14. An arranging device according to claim 11, wherein said one or more plates comprise one or more spacers that space two or more of said plurality of hooks.

15. A guide for guiding a plurality of anastomotic hooks into a blood vessel, said guide comprising:
a guide tip adapted to be placed through an aperture in a blood vessel, said tip being further adapted to guide a plurality of hooks into said blood vessel without contacting said aperture; and
at least one guide wall attached to said tip, said at least one wall being adapted to guide said plurality of hooks toward said tip while protecting said hooks from contacting tissue in proximity to said aperture.

16. A guide according to claim 15, wherein said tip is adapted to protect said hooks from contacting the edges of said aperture during said guiding.

17. A guide according to claim 15, wherein said tip comprises a blunt end.

18. A guide according to claim 15, wherein said tip comprises a sharp end adapted to form said aperture.

19. A guide according to claim 15, wherein said at least one wall comprises two or more walls.

20. A guide according to claim 19, wherein said two or more walls are connected to each other.

21. A guide according to claim 15, wherein said guide is adapted to remove from said blood vessel following guiding said plurality of anastomotic hooks.

22. A guide according to claim 15, wherein said guide is rotatably mounted on an anastomotic connector delivery system, such that said hooks selectively enter said guide by said rotation.

23. A hook grasper for retractably grasping a plurality of anastomotic connector hooks, comprising:
a handle;
at least one grasping wire projecting from said handle, adapted to grasp and compress a plurality of anastomotic connector hooks; and
a grasping wire controller that controls extension of said extension wire in relation to said handle, such that at one extension, the grasping wire receives uncompressed hooks, at a second extension the grasping wire compresses the hooks and at a third, further extension, the hooks are released.

24. A hook grasper according to claim 23, wherein said adaptation to grasp a plurality of hooks comprises a curvature adapted for partially encircling a plurality of said hooks.

25. A hook grasper according to claim 23, wherein said adaptation to grasp a plurality of hooks comprises a form of a loop.

26. A hook grasper according to claim 25, wherein said wire defines at least one breakaway area on said wire that breaks when said wire is pulled away taunt.

27. A hook grasper according to claim 25, wherein said extensions comprises progressive retraction positions of said wire.

28. A hook grasper according to claim 25, mounted on a connector delivery system.

29. A hook grasper according to claim 25, comprising a tube into which said wire is retracted between the extension position.

30. A blood vessel cutter for cutting an aperture in a blood vessel, comprising:
a handle having a longitudinal axis; and
a horn-shaped cutting edge connect to said handle, said cutting edge describing an arc around said longitudinal axis upon rotation of said handle around said longitudinal axis;
whereby, said cutting edge is adapted to cut an aperture in a blood vessel when held proximate to said vessel during said rotation.

31. An anastomotic delivery system for delivering an anastomotic connector into a blood vessel and tearing one or more extensions off of said connector, said system comprising:
a puller which is coupled to said extensions;
a manual input operative to retract said puller;
a loaded spring, coupled to said puller;
a selectable interlock which selectively prevents a release of said spring; and
an interlock release, coupled to said manual input, and operative to release said interlock depending on a retraction of said puller, wherein releasing said interlock releases said spring to tear said extensions.

32. A system according to claim 31, comprising a shock absorber to reduce a delivery of shock from said spring to a housing of said system, when said spring is released.

33. A rotatable anastomotic connector delivery system, comprising:
a handle; and
an anastomotic connector holder rotatably attached to said handle.

34. A rotatable system according to claim 33, wherein said rotator comprises one or more rotational extent rests.

35. A rotatable system according to claim 33, wherein said device comprises a rotational extent indicator.

36. A two part anastomosis delivery system, comprising:
a handle section, adapted to apply force sufficient to deploy an anastomotic connector, through a coupling thereof; and
a capsule adapted to be removably mounted on said handle and to apply said force through said coupling to a connector mounted on said capsule.

37. A system according to claim 36, wherein said capsule is rotatably mounted on said handle section.

38. A system according to claim 36, wherein said capsule includes a hook retractor operative to manually retract and extend hooks of said connector.

39. A system according to claim 38, wherein said capsule comprises two axial sections which rotate one relative to the other to effect said extension and retraction.

40. A system according to claim 38, wherein said capsule comprises an axially moving pin which effects said extension and retraction.

41. A system according to claim 38, wherein said capsule comprises a pin which rotates around an axis of said capsule to effect said extension and retraction.

42. A system according to claim 38, wherein said handle section prevents access to said hook retractor, when said capsule is mounted on said handle.

43. A system according to claim 38, wherein said capsule is adapted to not mount on said handle section if said hooks are not in a pre-defined axial position.

44. A system according to claim 38, wherein said capsule comprises a stop which restricts axial motion of said hooks.

45. An anastomotic connector for attaching two blood vessels comprising:
a plurality of eye segments, each defining a channel and each including a part of an interlock mechanism on said channel; and
a plurality of hook segments, each defining a tissue holding area, each adapted to pass through said channel and including a second part of said interlock mechanism, wherein, said interlock mechanism engages for a hook and an eye segment when said hook segment is retracted back into said eye segment enough to attach two layers of vascular tissue between said eye segment and said hook segment.

46. A connector according to claim 45, wherein said hook segment comprises a curved hook tip having a sharpened tissue penetrating tip at its end.

47. A connector according to claim 46, wherein said tip is generally aligned with a center of said eye segment.

48. A connector according to claim 47, wherein said eye segment defines an aperture aligned with said tip.

49. A connector according to claim 48, wherein said eye segment comprises at least one flap in said aperture, to reduce tissue ingress into said aperture.

50. A connector according to claim 45, wherein said eye segment comprises a body of a closed ring.

51. A connector according to claim 45, wherein said eye segment comprises a body which is open at at least one point of its circumference.

52. A connector according to claim 45, wherein said interlocking mechanism is stiff enough and strong enough to hold said hook segment while it is being torn off an extension thereof, by pulling on the extension.

53. A connector according to claim 45, wherein said hook segment includes an extension which is torn off said hook segment during deployment by pulling, said hook segment defining a rest stop where said hook segment is held during said pulling.

54. A connector according to claim 53, wherein said extension defines a slot terminating at said rest stop.

55. A connector according to claim 45, wherein said interlocking mechanism is substantially all on a plane of said eye segment, once interlocked.

56. A connector according to claim 45, wherein said interlocking mechanism comprises at least one tab that is perpendicular to an axis of said hook segment, at said channel.

57. A connector according to claim 56, wherein said tab enters a matching aperture formed in said hook segment.

58. A connector according to claim 56, wherein said tab transfixes a matching aperture formed in said hook segment.

59. A connector according to claim 56, wherein said tab transfixes a matching open slot formed in said hook segment.

60. A connector according to claim 56, comprising at least one spring element which approximates said channel and said tab.

61. A connector according to claim 60, wherein said tab is mounted on said spring element.

62. A connector according to claim 60, wherein said tab is not mounted on said spring element.

63. A connector according to claim 62, wherein said spring element urges said hook element against said tab.

64. A connector according to claim 60, wherein said spring element is formed of an outer portion of said eye segment.

65. A connector according to claim 60, wherein said spring element is attached to said eye segment near said channel.

66. A connector according to claim 60, wherein said spring element is attached to said eye segment far from said channel.

67. A connector according to claim 60, wherein said eye segment includes a support bar on which said spring element is attached.

68. A connector according to claim 60, wherein said at least one tab comprises only a single tab.

69. A connector according to claim 60, wherein said at least one tab comprises at least two tabs.

70. A connector according to claim 60, wherein said hook element includes an extension which is torn off during deployment, said extension defining an alternative aperture for locking said tab spaced from said tissue holding area.

71. A connector according to claim 45, wherein said eye segments are interconnected after deployment.

72. A connector according to claim 45, wherein said eye segments are not interconnected after deployment.

73. An anastomotic connection clip element, comprising:
a base;
at least one stitching spike, attached to said base and having a sharp end adapted to penetrate vascular tissue; and
at least one top spike, attached to said base,
wherein said stitching spike and said top spike diverge in opposite directions near said base and curve back towards each other away from said base.

74. An anastomotic connection clip element according to claim 73, wherein said spikes comprises at least one spike of one type and two spikes of the other type, interleaved.

75. An anastomotic connection clip element according to claim 74, wherein said top spike is curved to conform to a blood vessel curvature.

76. An anastomotic connection clip element according to claim 74, wherein said element is coupled to a plurality of clip elements, to form a connector.

77. A method of guiding a plurality of anastomotic hooks into a blood vessel, comprising:
inserting a tip of a guide into an aperture in a blood vessel, while a portion of the guide remains outside the blood vessel; and
guiding the hooks along the guide into the blood vessel without contacting said aperture, in a manner which protects the hooks from contacting tissue in proximity to said aperture.

78. A method according to claim 77, wherein said tip is adapted to protect said hooks from contacting the edges of said aperture during said guiding.

79. A method according to claim 77, comprising forming the aperture using the tip of the guide.

80. A method according to claim 77, wherein guiding the hooks along the guide comprises guiding the hooks within a groove of the guide having two or more walls.

81. A method according to claim 77, comprising removing the guide from the blood vessel following the guiding of the hooks into the blood vessel.

82. An anastomotic connector delivery system, comprising:
a hook holder adapted to hold a plurality of anastomotic hooks;
a handle attached to the hook holder in a manner which allows an operator to move the hook holder toward a blood vessel; and
a protection guide, mounted on the hook holder or the handle, rotatably relative to the hook holder, such that in a first position the protection guide is located between hooks held by the hook holder and the blood vessel and in a second position allows the hooks to move into an aperture in the blood vessel.

83. A delivery system according to claim 82, wherein the protection guide has an arc shape.

84. A delivery system according to claim 82, wherein the protection guide has a spiral shape.

85. A delivery system according to claim 82, wherein the guide includes two or more walls connected to each other.

86. A transfixing assistance device according to claim 1, wherein said handle is longer than the extension.

87. A transfixing assistance device according to claim 1, wherein the end of the extension is not substantially parallel the longitudinal axis.
